(19) United States
(12) Patent Application Publication
(10) Pub. No.: US 2024/0353426 A1
(43) Pub. Date: Oct. 24, 2024

(54) Methods for Diagnosing and Treating Ischemic Eye Disease

(71) Applicant: The Board of Trustees of the Leland Stanford Junior University, Stanford, CA (US)

(72) Inventors: Vinit Mahajan, Los Altos, CA (US); Gabriel Velez, Palo Alto, CA (US); Cassie Ann Ludwig, Palo Alto, CA (US)

(21) Appl. No.: 18/683,069

(22) PCT Filed: Aug. 11, 2022
(86) PCT No.: PCT/US2022/074856

§ 371 Date: Feb. 12, 2024

Related U.S. Application Data
(60) Provisional application No. 63/232,850, filed on Aug. 13, 2021.

(51) Int. Cl. G01N 33/68; A61K 31/155; A61K 31/573; A61K 38/17; A61K 39/00

(52) U.S. Cl. CPC G01N 33/6893; A61K 31/155; A61K 31/573; A61K 38/179; A61K 2039/505; G01N 2800/042; G01N 2800/16; G01N 2800/52

(57) ABSTRACT

Compositions, methods, and kits are provided for diagnosing and treating proliferative diabetic retinopathy. The identified biomarkers can be used alone or in combination with one or more additional biomarkers or relevant clinical parameters in prognosis, diagnosis, or monitoring treatment of proliferative diabetic retinopathy. Methods of treating a subject for proliferative diabetic retinopathy are also provided.

### BACKGROUND OF THE INVENTION

Diabetic retinopathy is the leading cause of vision loss in adults 20 to 64 years of age (Ding et al., Curr Diab Rep. 2012; 12(4):346-354). An estimated one third of the population will develop diabetic retinopathy, and one third will progress to vision threatening stages including diabetic macular edema (DME) and proliferative diabetic retinopathy (PDR) (Zheng et al., Indian J Ophthalmol. 2012; 60(5):428-431. Chronically elevated intravascular glucose levels drive reactive oxygen species (ROS) production, which causes endothelial cell and pericyte loss and senescence that lead to leakage of plasma and erythrocytes into the surrounding retinal tissue (Wert et al., Free Radic Biol Med. 2018; 124:408-419). This results in the buildup of intraretinal fluid (DME) and hemorrhage. Failure of leaky vessels to provide sufficient oxygenation can result in subsequent retinal ischemia leading to abnormal growth of new blood vessels in an attempt to recover oxygenation. Angiogenesis is driven in part by vascular endothelial growth factor (VEGF) from vascular pericytes, retinal ganglion cells, and glia (Nita et al., Oxid Med Cell Longev. 2016; 2016:3164734). Unfortunately, the body's attempt at correcting hypoxia can result in vision loss as new blood vessel formation in pathologic locations (i.e., PDR) may result in vitreous hemorrhage (VH) and tractional retinal detachments (TRDs) (Frank et al., N Engl J Med. 2004; 350(1):48-58). In PDR, retinal ischemia drives pathological neovascularization with subsequent vitreous and retinal hemorrhage, retinal fibrosis, and tractional retinal detachment. Current treatment includes destructive laser photocoagulation of the peripheral retina and intravitreal injections of anti-VEGF antibodies. Despite these treatments, there are no interventions that successfully prevent the formation of proliferative fibrotic membranes that cause tractional retinal detachment.

### SUMMARY OF THE INVENTION

Compositions, methods, and kits are provided for diagnosing and treating proliferative diabetic retinopathy. The identified biomarkers can be used alone or in combination with one or more additional biomarkers or relevant clinical parameters in prognosis, diagnosis, or monitoring treatment of proliferative diabetic retinopathy. Methods of treating a subject for proliferative diabetic retinopathy are also provided.

Biomarkers that can be used in diagnosing proliferative diabetic retinopathy include, without limitation, interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL).

In certain embodiments, a panel of biomarkers is used for diagnosis of PDR. Biomarker panels of any size can be used in the practice of the subject methods. Biomarker panels for diagnosing PDR typically comprise at least 3 biomarkers and up to 37 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or more biomarkers. In some embodiments, the biomarker panel comprises or consists of all of the biomarkers: interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL). Although smaller biomarker panels are usually more economical, larger biomarker panels (i.e., greater than 20 biomarkers) have the advantage of providing more detailed information and can also be used in the practice of the subject methods.

In one aspect, a method of diagnosing and treating PDR in a patient is provided, the method comprising: a) obtaining a vitreous sample from an eye of the patient; b) measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the vitreous sample, wherein decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) compared to reference value ranges for a vitreous sample from a control subject, and increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) compared to reference value ranges for a vitreous sample from a control subject indicate that the patient has PDR; and c) treating the patient for the PDR, if the patient has a positive diagnosis for PDR.

In certain embodiments, the levels of expression of at least two, at least three, or at least four biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) are measured in the vitreous sample.

In certain embodiments, the levels of expression of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) are measured in the vitreous sample.

In certain embodiments, treating the patient for the PDR comprises administering a corticosteroid such as, but not limited to, triamcinolone or fluocinolone acetonide; and/or administering a vascular endothelial growth factor inhibitor such as, but not limited to, bevacizumab, aflibercept, or ranibizumab; and/or performing laser surgery such as, but not limited to, photocoagulation or panretinal photocoagulation; and/or performing a vitrectomy.

In another aspect, a method of monitoring PDR in a patient is provided, the method comprising: a) obtaining a first vitreous sample from an eye of the patient at a first time point and a second vitreous sample from the eye of the subject later at a second time point; b) measuring one or more biomarkers in the first vitreous sample and the second vitreous sample, wherein the biomarkers are selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL); and c) analyzing the levels of expression of the one or more biomarkers in conjunction with respective reference value ranges for said biomarkers, wherein detection of decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and detection of increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening, and detection of increased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and decreased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL in the second vitreous sample compared to the first vitreous sample indicate that the patient is improving.

In another aspect, a method of monitoring efficacy of a treatment of a patient PDR is provided, the method comprising: a) obtaining a first vitreous sample from the patient before the patient undergoes the treatment and a second vitreous sample from the subject after the patient undergoes the treatment; b) measuring one or more biomarkers in the first vitreous sample and the second vitreous sample, wherein the biomarkers are selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL); and c) evaluating the efficacy of the treatment, wherein detection of decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and detection of increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening or not responding to the treatment, and detection of increased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and decreased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL in the second vitreous sample compared to the first vitreous sample indicate that the patient is improving. In certain embodiments, the method further comprises altering the treatment if the patient is worsening or not responding to the treatment.

In another aspect, a kit comprising agents for detecting at least 3 biomarkers selected from the group consisting of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) is provided.

In certain embodiments, the kit comprises agents for detecting a biomarker panel comprising or consisting of all of the interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) biomarkers.

In certain embodiments, the kit, further comprises reagents for performing an immunoassay.

In certain embodiments, the kit comprises an antibody that specifically binds to interleukin 12 (IL-12p70), an antibody that specifically binds to thyroid-stimulating hormone (TSH), an antibody that specifically binds to interleukin-1 family member 6 (IL-1 F6), an antibody that specifically binds to fibroblast growth factor 6 (FGF-6), an antibody that specifically binds to bone morphogenetic protein 7 (BMP-7), an antibody that specifically binds to interleukin 1 beta (IL-1b), an antibody that specifically binds to follicle-stimulating hormone (FSH), an antibody that specifically binds to tafazzin (TAZ), an antibody that specifically binds to interleukin 34 (IL-34), an antibody that specifically binds to carcinoma antigen 15-3 (CA15-3), an antibody that specifically binds to albumin (ALB), an antibody that specifically binds to cadherin-6 (CDH6), an antibody that specifically binds to semaphorin 6D (SEMA6D), an antibody that specifically binds to tenascin R (TNR), an antibody that specifically binds to tumor necrosis factor receptor superfamily member 9 (4-1 BB), an antibody that specifically binds to vascular endothelial growth factor (VEGF), an antibody that specifically binds to galectin-2 (LGALS2), an antibody that specifically binds to transmembrane activator and CAML interactor (TACI), an antibody that specifically binds to monocyte chemoattractant protein 1 (MCP-1), an antibody that specifically binds to angiopoietin 2 (ANG-2), an antibody that specifically binds to interferon gamma-induced protein 10 (IP-10), an antibody that specifically binds to galectin-3 (LGALS3), an antibody that specifically binds to plasminogen activator inhibitor-1 (PAI-1), an antibody that specifically binds to C—C motif chemokine ligand 4 (MIP-1b), an antibody that specifically binds to cluster of differentiation 97 (CD97), an antibody that specifically binds to platelet-derived growth factor having 2 A subunits (PDGF-AA), an antibody that specifically binds to regenerating family member 4 (REG4), an antibody that specifically binds to nucleosome assembly protein 1 like 4 (NAP-2), an antibody that specifically binds to ferritin, matrix metallopeptidase 13 (MMP-13), an antibody that specifically binds to cluster of differentiation 99 (CD99), an antibody that specifically binds to B cell maturation antigen (BCMA), an antibody that specifically binds to cathepsin S (CTSS), an antibody that specifically binds to T cell immunoglobulin mucin 3 (TIM-3), midkine (MDK), an antibody that specifically binds to glycoxalase II, and an antibody that specifically binds to mannose-binding lectin (MBL).

In certain embodiments, the kit further comprising instructions for determining whether a subject has proliferative diabetic retinopathy (PDR).

In another aspect, a protein selected from the group consisting of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) for use as a biomarker in diagnosing proliferative diabetic retinopathy (PDR) is provided.

In another aspect, an in vitro method of diagnosing proliferative diabetic retinopathy (PDR) is provided, the method comprising: a) obtaining a vitreous sample from an eye of the patient; b) measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the vitreous sample, wherein decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) compared to reference value ranges for a vitreous sample from a control subject, and increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) compared to reference value ranges for a vitreous sample from a control subject indicate that the patient has PDR.

In certain embodiments, measuring the level of expression of a biomarker comprises measuring a level of expression of a protein. For example, levels of a biomarker protein may be measured by a method including, but not limited to, mass spectrometry or tandem mass spectrometry, an enzymatic or biochemical assay, liquid chromatography, NMR, or an immunoassay, such as an enzyme-linked immunosorbent assay (ELISA), a radioimmunoassay (RIA), an immunofluorescent assay (IFA), immunohistochemistry, fluorescence-activated cell sorting (FACS), or a Western Blot.

In another aspect, a method of treating a subject for proliferative diabetic retinopathy is provided, the method comprising administering a therapeutically effective amount of pimagedine, atacicept, or an ANG-2 inhibitor to the subject. In some embodiments, the ANG-2 inhibitor is trebananib, tebastanib, or MEDI3617. Any suitable mode of administration may be used. For example, the pimagedine, atacicept, or ANG-2 inhibitor is administered locally to the eye. In some embodiments, the pimagedine, atacicept, or ANG-2 inhibitor is administered locally to the retina. Alternatively, the pimagedine, atacicept, or ANG-2 inhibitor may be administered intravitreally, intravenously, subcutaneously, or orally.

In certain embodiments, multiple therapeutically effective doses of the pimagedine, atacicept, or ANG-2 inhibitor are administered to the subject. For example, the pimagedine, atacicept, or ANG-2 inhibitor may be administered daily or intermittently.

In another aspect, a composition comprising pimagedine, atacicept, or an ANG-2 inhibitor for use in a method of treating proliferative diabetic retinopathy is provided. In some embodiments, the ANG-2 inhibitor is trebananib, tebastanib, or MEDI3617.

In another aspect, a composition comprising pimagedine, atacicept, or an ANG-2 inhibitor for use as a medicament for the treatment of proliferative diabetic retinopathy is provided. In some embodiments, the ANG-2 inhibitor is trebananib, tebastanib, or MEDI3617.

### BRIEF DESCRIPTION OF THE DRAWINGS

FIGS. 1A-1B. Principal component analysis (PCA) of the proteomics of proliferative diabetic retinopathy (PDR): (FIG. 1A) The score plot of PC1 and PC2 showed separation between the 6 PDR cases (red) and 6 controls with epiretinal membrane (ERM) (blue) based on differentially expressed proteins that were significantly different between the two groups. (FIG. 11B) Volcano plot representing the protein intensities analyzed by 1-way ANOVA. The x-axis displays the noise-adjusted signal as the log 2 fold-change value (PDR vs. controls) and the y-axis displays the noise-adjusted signal as the −log 10 (p-value) from the 1-way ANOVA analysis.

FIG. 2. Differentially expressed proteins reveal differences between proliferative diabetic retinopathy (PDR) cases and epiretinal membrane (ERM) controls: Protein concentrations from the multiplex ELISA array were analyzed for differentially expressed proteins. A total of 37 proteins were differentially expressed among ERM and PDR samples (22 upregulated proteins in PDR samples and 15 downregulated proteins; p<0.01). Results are represented as a heatmap and display protein expression levels on a logarithmic scale. Orange indicates high expression while dark green/black indicates low or no expression.

FIG. 3. Protein-protein interaction analysis and drug repositioning: Protein-protein interaction analysis demonstrates interaction between 41 proteins differentially expressed in PDR. Proteins separated by involvement in fibrosis, angiogenesis, chemotaxis, inflammation, immune development, and cell regulation and metabolism. Drugs that may inhibit each protein's function are noted.

FIGS. 4A-4C. Gene ontology and pathway analysis reveals differences between proliferative diabetic retinopathy (PDR) cases and epiretinal membrane (ERM) controls: (FIG. 4A) Differentially expressed proteins (P<0.05) from PDR cases compared to controls. Gene ontology analysis categorizing protein groups by biological process, molecular function, and cellular compartment. A greater proportion of immunologic proteins, proteins involved in catalytic activity and proteins located in the extracellular region is seen in PDR cases. Top twenty pathways from the (FIG. 4B) KEGG and (FIG. 4C) PANTHER databases represented in PDR. Pathways are ranked by −log (p-value) obtained from the right-tailed Fisher's Exact Test.

### DETAILED DESCRIPTION OF THE INVENTION

Compositions, methods, and kits are provided for diagnosing and treating proliferative diabetic retinopathy. The identified biomarkers can be used alone or in combination with one or more additional biomarkers or relevant clinical parameters in prognosis, diagnosis, or monitoring treatment of proliferative diabetic retinopathy. Methods of treating a subject for proliferative diabetic retinopathy are also provided.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g. peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Biomarkers. The term “biomarker” as used herein refers to a compound, such as a protein, a polypeptide, a peptide, a mRNA, a metabolite, or a metabolic byproduct which is differentially expressed or present at different concentrations, levels or frequencies in one sample compared to another, such as a vitreous sample from patients who have PDR compared to a vitreous sample from healthy control subjects (i.e., subjects not having PDR or other eye disease). Biomarkers include, but are not limited to, interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL).

In some embodiments, the concentration or level of a biomarker is determined before and after the administration of a treatment to a patient. The treatment may comprise, for example, without limitation, administering a corticosteroid or a vascular endothelial growth factor inhibitor or performing laser surgery or a vitrectomy. The degree of change in the concentration or level of a biomarker, or lack thereof, is interpreted as an indication of whether the treatment has the desired effect (e.g., decreasing proliferation of blood vessels, preventing or reducing damage to the retina and loss of vision). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in vitreous samples (e.g., immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “PDR” biomarker expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Vitreous sample. The term “vitreous sample” with respect to an individual encompasses samples taken from the vitreous humor extracellular matrix located in the posterior chamber of the eye, such as a surgical or biopsy specimen isolated therefrom. Vitreous samples can be obtained by any suitable method such as by surgical resection or by biopsy, for example, using fine needle aspiration (FNA) or pars plana vitrectomy (PPV). The definition also includes samples that have been manipulated in any way after their procurement, such as by treatment with reagents; washed; or enriched for particular types of molecules, e.g., proteins, peptides, etc.

Obtaining and assaying a sample. The term “assaying” is used herein to include the physical steps of manipulating a vitreous sample to generate data related to the vitreous sample. As will be readily understood by one of ordinary skill in the art, a vitreous sample must be “obtained” prior to assaying the sample. Thus, the term “assaying” implies that the sample has been obtained. The terms “obtained” or “obtaining” as used herein encompass the act of receiving an extracted or isolated vitreous sample. For example, a testing facility can “obtain” a vitreous sample in the mail (or via delivery, etc.) prior to assaying the sample. In some such cases, the vitreous sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a vitreous sample from a subject. Accordingly, a vitreous sample can be isolated from a subject (and thus “obtained”) by the same person or same entity that subsequently assays the sample. When a vitreous sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a vitreous sample.

In some embodiments, the step of obtaining comprises the step of isolating a vitreous sample (e.g., a pre-treatment vitreous sample, a post-treatment vitreous sample, etc.). Methods and protocols for isolating various vitreous samples will be known to one of ordinary skill in the art and any convenient method may be used to isolate a vitreous sample.

It will be understood by one of ordinary skill in the art that in some cases, it is convenient to wait until multiple samples (e.g., a pre-treatment vitreous sample and a post-treatment vitreous sample) have been obtained prior to assaying the samples. Accordingly, in some cases an isolated vitreous sample (e.g., a pre-treatment vitreous sample, a post-treatment vitreous sample, etc.) is stored until all appropriate samples have been obtained. One of ordinary skill in the art will understand how to appropriately store a variety of different types of vitreous samples and any convenient method of storage may be used (e.g., refrigeration) that is appropriate for the particular vitreous sample. In some embodiments, a pre-treatment vitreous sample is assayed prior to obtaining a post-treatment vitreous sample. In some cases, a pre-treatment vitreous sample and a post-treatment vitreous sample are assayed in parallel. In some cases, multiple different post-treatment vitreous samples and/or a pre-treatment vitreous sample are assayed in parallel. In some cases, vitreous samples are processed immediately or as soon as possible after they are obtained.

In some embodiments, the concentration (i.e., “level”), or expression level of a gene product, which may be a protein, peptide, etc., (which will be referenced herein as a biomarker), in a vitreous sample is measured (i.e., “determined”). By “expression level” (or “level”) it is meant the level of gene product (e.g., the absolute and/or normalized value determined for the RNA expression level of a biomarker or for the expression level of the encoded polypeptide, or the concentration of the protein in a vitreous sample). The term “gene product” or “expression product” are used herein to refer to the RNA transcription products (RNA transcripts, e.g., mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple vitreous samples from the same individual (e.g., vitreous samples taken at different points in time from the same individual). This is because the units cancel when calculating a fold-change (i.e., determining a ratio) in the expression level from one vitreous sample to the next (e.g., vitreous samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

For measuring protein levels, the amount or level of a protein in the vitreous sample is determined. In some cases, the protein comprises a post-translational modification (e.g., phosphorylation, glycosylation) associated with regulation of activity of the protein such as by a signaling cascade, wherein the modified protein is the biomarker, and the amount of the modified protein is therefore measured. In some embodiments, an extracellular protein level is measured. For example, in some cases, the protein (i.e., polypeptide) being measured is a secreted protein (e.g., extracellular matrix protein) and the concentration can therefore be measured in vitreous fluid. In some embodiments, concentration is a relative value measured by comparing the level of one protein relative to another protein. In other embodiments the concentration is an absolute measurement of weight/volume or weight/weight.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different manners of assaying for protein levels are known to one of ordinary skill in the art and any convenient method may be used, one representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### Additional Terms

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with PDR) as well as those in which prevention is desired (e.g., those with diabetes (type I or type 1l), those with a genetic predisposition to developing PDR, those with increased susceptibility to PDR, those suspected of having PDR, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Substantially purified” generally refers to isolation of a component such as a substance (compound, drug, inhibitor, metabolite, nucleic acid, polynucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of vitreous sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have PDR, a recommendation for treatment, and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Biomarkers and Diagnostic Methods

Biomarkers that can be used in the practice of the subject methods include, without limitation, interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL). Differential expression of these biomarkers is associated with proliferative diabetic retinopathy and therefore expression profiles of these biomarkers are useful for diagnosing proliferative diabetic retinopathy.

In certain embodiments, a panel of biomarkers is provided for diagnosis of proliferative diabetic retinopathy. Biomarker panels of any size can be used in the practice of the subject methods. Biomarker panels for diagnosing proliferative diabetic retinopathy typically comprise at least 3 biomarkers and up to 20 biomarkers, including any number of biomarkers in between, such as 3, 4, 5, 6, 7, 8, 9, 10, 11, 12, 13, 14, 15, 16, 17, 18, 19, or 20 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20, or more biomarkers. In some embodiments, the biomarker panel comprises or consists of all of the interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) biomarkers. Although smaller biomarker panels are usually more economical, larger biomarker panels (i.e., greater than 20 biomarkers) have the advantage of providing more detailed information and can also be used in the practice of the subject methods.

A vitreous sample comprising the expressed biomarkers is obtained from the subject. The sample is taken from the vitreous humor extracellular matrix located in the posterior chamber of the eye of the subject. A “control” sample, as used herein, refers to a vitreous sample from a subject that is not diseased. That is, a control sample is obtained from a normal or healthy subject (e.g., an individual known to not have proliferative diabetic retinopathy or other eye disease). A vitreous sample can be obtained from a subject by conventional techniques. For example, vitreous samples can be obtained by surgical resection or by biopsy using fine needle aspiration (FNA) or pars plana vitrectomy (PPV) according to methods well known in the art.

When analyzing the levels of biomarkers in a vitreous sample from a subject, the reference value ranges used for comparison can represent the levels of one or more biomarkers in a vitreous sample from one or more subjects without proliferative diabetic retinopathy (i.e., normal or healthy control). Alternatively, the reference values can represent the levels of one or more biomarkers from one or more subjects with proliferative diabetic retinopathy, wherein similarity to the reference value ranges indicates the subject has proliferative diabetic retinopathy. More specifically, the reference value ranges can represent the levels of one or more biomarkers from one or more subjects with proliferative diabetic retinopathy (a “proliferative diabetic retinopathy” biomarker expression profile).

Accordingly, in one aspect, a method of diagnosing proliferative diabetic retinopathy in a patient is provided, the method comprising: a) obtaining a vitreous sample from an eye of the patient; and b) measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the vitreous sample, wherein differential expression of the of one or more biomarkers selected from the group consisting of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) compared to reference value ranges for a vitreous sample from a control subject indicate that the patient has proliferative diabetic retinopathy.

In certain embodiments, the levels of expression of the vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) biomarkers in the vitreous sample from the eye of the patient are measured, wherein increased levels of expression of the vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) biomarkers in the vitreous sample from the eye of the patient compared to reference value ranges for the biomarkers in a vitreous sample from a control subject indicate that the patient has proliferative diabetic retinopathy.

In certain embodiments, the levels of expression of the interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) biomarkers in the vitreous sample from the eye of the patient are measured, wherein decreased levels of expression of the interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) biomarkers in the vitreous sample from the eye of the patient compared to reference value ranges for the biomarkers in a vitreous sample from a control subject indicate that the patient has proliferative diabetic retinopathy.

The methods described herein may be used to determine an appropriate treatment regimen for a patient and, in particular, whether a patient should be treated for proliferative diabetic retinopathy. For example, a patient is selected for treatment for proliferative diabetic retinopathy if the patient has a positive diagnosis for proliferative diabetic retinopathy based on a biomarker expression profile, as described herein. The treatment for proliferative diabetic retinopathy may comprise, for example, laser surgery, laser coagulation such as focal laser treatment (photocoagulation) or scatter laser treatment (panretinal photocoagulation), vitrectomy, or local administration of corticosteroids (e.g., triamcinolone or fluocinolone acetonide) or vascular endothelial growth factor inhibitors (e.g., ranibizumab, aflibercept, or bevacizumab) into the eye, for example, by injection or with an intravitreal implant (e.g., fluocinolone acetonide intravitreal implant (Iluvien®, Alimera Sciences, Alpharetta, GA). In cases in which disease is not severe, for example, if the subject has mild to moderate nonproliferative diabetic retinopathy, the subject may be treated with more careful diabetes management, such as better controlling blood glucose levels or adjusting diet to reduce sugar and carbohydrate intake, followed up with continued monitoring of the eyes for disease progression. For more severe cases, in which the subject is diagnosed with proliferative diabetic retinopathy, the subject may be treated, for example, with laser surgery, laser coagulation, vitrectomy, or injection of corticosteroids (e.g., triamcinolone) or vascular endothelial growth factor inhibitors (e.g., ranibizumab) into the eye.

In some embodiments, the methods described herein are used for monitoring proliferative diabetic retinopathy in a subject. For example, a first vitreous sample can be obtained from the patient at a first time point and a second vitreous sample can be obtained from the subject at a second (later) time point. In one embodiment, proliferative diabetic retinopathy is monitored in the patient by measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the first vitreous sample and the second vitreous sample; and analyzing the levels of expression of the one or more biomarkers in conjunction with respective reference value ranges for the biomarkers, wherein detection of decreased levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and detection of increased levels of expression of one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening (i.e., diabetic retinopathy is progressing), and detection of increased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and detection of decreased levels of expression of one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is improving.

The subject methods may also be used for assaying pre-treatment and post-treatment vitreous samples obtained from an individual to determine whether the individual is responsive or not responsive to a treatment. For example, a first vitreous sample can be obtained from a subject before the subject undergoes the therapy, and a second vitreous sample can be obtained from the subject after the subject undergoes the therapy. In one embodiment, the efficacy of a treatment of a patient for proliferative diabetic retinopathy is monitored by measuring one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the first vitreous sample and the second vitreous sample; and evaluating the efficacy of the treatment, wherein detection of decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and detection of increased levels of expression of one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening or not responding to the treatment, and detection of increased levels of expression of the one or more biomarkers selected from the group consisting of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and detection of decreased levels of expression of one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is improving.

In some cases, the diagnostic methods described herein may be used by themselves or combined with medical imaging or other ophthalmology techniques for detecting ocular lesions to confirm the diagnosis and further evaluate the severity and extent of disease (e.g., detect retina damage, leaking, neovascularization, or other abnormalities of retinal arteries and veins, and/or macular edema) to aid in determining prognosis and evaluating optimal strategies for treatment (e.g., laser surgery, laser coagulation, vitrectomy, or injection of corticosteroids (e.g., triamcinolone) or vascular endothelial growth factor inhibitors (e.g., ranibizumab) into the eye, etc.). Exemplary medical imaging and ophthalmology techniques include, without limitation, fundus photography, fluorescein angiography, retinal vessel analysis, ultrasonography, optical coherence tomography, autofluorescence, indocyanine green angiography, and the radioactive phosphorus uptake test on the eye.

In some cases, combinations of biomarkers are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers can be used in any combination. In other embodiments, all the biomarkers are used. The quantitative values may be combined in linear or non-linear fashion to calculate one or more risk scores for proliferative diabetic retinopathy for the individual.

The level of a biomarker in a pre-treatment vitreous sample can be referred to as a “pre-treatment value” because the first vitreous sample is isolated from the individual prior to the administration of the therapy (i.e., “pre-treatment”). The level of a biomarker in the pre-treatment vitreous sample can also be referred to as a “baseline value” because this value is the value to which “post-treatment” values are compared. In some cases, the baseline value (i.e., “pre-treatment value”) is determined by determining the level of a biomarker in multiple (i.e., more than one, e.g., two or more, three or more, for or more, five or more, etc.) pre-treatment vitreous samples. In some cases, the multiple pre-treatment vitreous samples are isolated from an individual at different time points in order to assess natural fluctuations in biomarker levels prior to treatment. As such, in some cases, one or more (e.g., two or more, three or more, for or more, five or more, etc.) pre-treatment vitreous samples are isolated from the individual. In some embodiments, all of the pre-treatment vitreous samples will be the same type of vitreous sample (e.g., a biopsy sample). In some cases, two or more pre-treatment vitreous samples are pooled prior to determining the level of the biomarker in the vitreous samples. In some cases, the level of the biomarker is determined separately for two or more pre-treatment vitreous samples and a “pre-treatment value” is calculated by averaging the separate measurements.

A post-treatment vitreous sample is isolated from an individual after the administration of a therapy. Thus, the level of a biomarker in a post-treatment sample can be referred to as a “post-treatment value”. In some embodiments, the level of a biomarker is measured in additional post-treatment vitreous samples (e.g., a second, third, fourth, fifth, etc. post-treatment vitreous sample). Because additional post-treatment vitreous samples are isolated from the individual after the administration of a treatment, the levels of a biomarker in the additional vitreous samples can also be referred to as “post-treatment values.”

The term “responsive” as used herein means that the treatment is having the desired effect such as improving vision, preventing, reducing or delaying vision loss, preventing or reducing retina damage, preventing or reducing neovascularization, and/or preventing or reducing macular edema. When the individual does not improve in response to the treatment, it may be desirable to seek a different therapy or treatment regime for the individual.

The determination that an individual has proliferative diabetic retinopathy by expression profiling is an active clinical application of the correlation between levels of a biomarker and the disease. For example, “determining” requires the active step of reviewing the data, which is produced during the active assaying step(s), and resolving whether an individual does or does not have proliferative diabetic retinopathy. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating proliferative diabetic retinopathy, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated for proliferative diabetic retinopathy by administering a vascular endothelial growth factor inhibitor. Then to “continue treatment” would be to continue with this type of treatment. If the current course of treatment is not effective, the treatment may be altered, e.g., switching treatment to a different vascular endothelial growth factor inhibitor or increasing the dose or frequency of administration of the vascular endothelial growth factor inhibitor, or changing to a different type of treatment such as laser surgery, laser coagulation, or vitrectomy.

In other words, the level of one or more biomarkers may be monitored in order to determine when to continue therapy and/or when to alter therapy. As such, a post-treatment vitreous sample can be isolated after any of the administrations and the vitreous sample can be assayed to determine the level of a biomarker. Accordingly, the subject methods can be used to determine whether an individual being treated for proliferative diabetic retinopathy is responsive or is maintaining responsiveness to a treatment.

The therapy can be administered to an individual any time after a pre-treatment vitreous sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment vitreous sample is isolated (or, when multiple pre-treatment vitreous samples are isolated, after the final pre-treatment vitreous sample is isolated).

In some cases, more than one type of therapy may be administered to the individual. For example, a subject who has proliferative diabetic retinopathy may be treated with a corticosteroid and a vascular endothelial growth factor inhibitor or laser surgery. A subject who has more severe disease or who is at high risk of disease progression, may be treated more aggressively. For example, treatment of a high-risk patient may include, without limitation, laser surgery, laser coagulation, or vitrectomy.

In some embodiments, the subject methods include providing an analysis indicating whether the individual is determined to have proliferative diabetic retinopathy. The analysis may further provide an analysis of whether an individual is responsive or not responsive to a treatment, or whether the individual is determined to be maintaining responsiveness or not maintaining responsiveness to a treatment for proliferative diabetic retinopathy. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in diagnosing a patient with proliferative diabetic retinopathy and determining the appropriate treatment for a subject, as well as monitoring responses of a subject to treatment.

In some embodiments, the amount or level in the sample of one or more proteins/polypeptides encoded by a gene of interest is determined. Any convenient protocol for evaluating protein levels may be employed where the level of one or more proteins in the assayed sample is determined. For antibody-based methods of protein level determination, any convenient antibody can be used that specifically binds to the intended biomarker such as interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL). The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

For measuring protein activity levels, the amount or level of protein activity in the sample of one or more proteins/polypeptides encoded by the gene of interest is determined.

In other embodiments, the amount or level in the sample of one or more proteins is determined. Any convenient method for measuring protein levels in a sample may be used, e.g., antibody-based methods, e.g., immunoassays, e.g., enzyme-linked immunosorbent assays (ELISAs), immunohistochemistry, and mass spectrometry.

The resultant data provides information regarding expression, amount, and/or activity for each of the biomarkers that have been measured, wherein the information is in terms of whether or not the biomarker is present (e.g., expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for proliferative diabetic retinopathy for an individual. In some embodiments, measurements for a biomarker or combinations of biomarkers are formulated into linear or non-linear models or algorithms (e.g., a ‘biomarker signature’) and converted into a likelihood score. This likelihood score indicates the probability that a vitreous sample is from a patient who may exhibit no evidence of disease, who may exhibit proliferative diabetic retinopathy. The models and/or algorithms can be provided in machine readable format, and may be used to correlate biomarker levels or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as having proliferative diabetic retinopathy. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Nave Bayes classifier, k-nearest neighbor, high-dimensional discriminant analysis, or Gaussian mixture models. In some instances, the machine learning algorithm comprises Random Forests.

### Kits

Also provided are kits for use in the methods, disclosed herein, for diagnosing PDR. The subject kits include agents (e.g., an antibody that specifically binds to a biomarker and/or other immunoassay reagents, and the like) for determining the level of at least one biomarker. In some embodiments, a kit comprises agents for determining the level of a single biomarker, two or more different biomarkers, three or more different biomarkers, or all the biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) biomarkers for diagnosing a patient with proliferative diabetic retinopathy.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

In certain embodiments, the kit further comprises reagents for performing an immunoassay. In some embodiments, the kit comprises at least two, at least three, at least four, or all the antibodies selected from an antibody that specifically binds to interleukin 12, an antibody that specifically binds to thyroid-stimulating hormone, an antibody that specifically binds to interleukin-1 family member 6, an antibody that specifically binds to fibroblast growth factor 6, an antibody that specifically binds to bone morphogenetic protein 7, an antibody that specifically binds to interleukin 1 beta, an antibody that specifically binds to follicle-stimulating hormone, an antibody that specifically binds to tafazzin, an antibody that specifically binds to interleukin 34, an antibody that specifically binds to carcinoma antigen 15-3, an antibody that specifically binds to albumin, an antibody that specifically binds to cadherin-6, an antibody that specifically binds to semaphorin 6D, an antibody that specifically binds to tenascin R, an antibody that specifically binds to tumor necrosis factor receptor superfamily member 9, an antibody that specifically binds to vascular endothelial growth factor, an antibody that specifically binds to galectin-2, an antibody that specifically binds to transmembrane activator and CAML interactor, an antibody that specifically binds to monocyte chemoattractant protein 1, an antibody that specifically binds to angiopoietin 2, an antibody that specifically binds to interferon gamma-induced protein 10 (IP-10), an antibody that specifically binds to galectin-3, an antibody that specifically binds to plasminogen activator inhibitor-1, an antibody that specifically binds to C—C motif chemokine ligand 4, an antibody that specifically binds to cluster of differentiation 97, an antibody that specifically binds to platelet-derived growth factor having 2 A subunits, an antibody that specifically binds to regenerating family member 4, an antibody that specifically binds to nucleosome assembly protein 1 like 4, an antibody that specifically binds to ferritin, an antibody that specifically binds to matrix metallopeptidase 13, an antibody that specifically binds to cluster of differentiation 99, an antibody that specifically binds to B cell maturation antigen, an antibody that specifically binds to cathepsin S, an antibody that specifically binds to T cell immunoglobulin mucin 3, an antibody that specifically binds to midkine, an antibody that specifically binds to glycoxalase 1l, and an antibody that specifically binds to mannose-binding lectin.

### Pharmaceutical Compositions

Pharmaceutical compositions comprising pimagedine, atacicept, or an ANG-2 inhibitor (e.g., trebananib, tebastanib, or MEDI3617) or a derivative thereof can be used to treat proliferative diabetic retinopathy. The pimagedine, atacicept, or an ANG-2 inhibitor (e.g., trebananib, tebastanib, or MEDI3617), or a derivative thereof, can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the pimagedine, atacicept, or an ANG-2 inhibitor (e.g., trebananib, tebastanib, or MEDI3617), or a derivative thereof, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the pimagedine, atacicept, or an ANG-2 inhibitor (e.g., trebananib, tebastanib, or MEDI3617), or a derivative thereof (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and, in particular, those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising pimagedine, atacicept, or an ANG-2 inhibitor (e.g., trebananib, tebastanib, or MEDI3617), or a derivative thereof are in unit dosage form, meaning an amount of a composition appropriate for a single dose, in a premeasured or pre-packaged form.

The compositions herein may optionally include one or more additional agents. Compounded preparations may include pimagedine, atacicept, or an ANG-2 inhibitor (e.g., trebananib, tebastanib, or MEDI3617), or a derivative thereof, and one or more other agents for treating proliferative diabetic retinopathy, such as, but not limited to, corticosteroids (e.g., triamcinolone and fluocinolone acetonide) and vascular endothelial growth factor inhibitors (e.g., ranibizumab, aflibercept, and bevacizumab); and the like.

Alternatively, such agents can be contained in a separate composition from the composition comprising the pimagedine, atacicept, or an ANG-2 inhibitor (e.g., trebananib, tebastanib, or MEDI3617), or the derivative thereof, and co-administered concurrently, before, or after the composition comprising the pimagedine, atacicept, or an ANG-2 inhibitor (e.g., trebananib, tebastanib, or MEDI3617) or the derivative thereof.

### Administration

At least one therapeutically effective cycle of treatment with a composition comprising pimagedine, atacicept, or an ANG-2 inhibitor (e.g., trebananib, tebastanib, or MEDI3617) will be administered to a subject for treatment of proliferative diabetic retinopathy. By “therapeutically effective dose or amount” of pimagedine, atacicept, or an ANG-2 inhibitor is intended an amount that, when administered, as described herein, brings about a positive therapeutic response, such as improved recovery from proliferative diabetic retinopathy. For example, a therapeutically effective dose or amount may improve vision, prevent, reduce, or delay vision loss, prevent or reduce retina damage, prevent or reduce neovascularization, and/or prevent or reduce macular edema. The exact amount required will vary from subject to subject, depending on the species, age, and general condition of the subject, the severity of the condition being treated, the particular type of pimagedine, atacicept, or ANG-2 inhibitor (e.g., trebananib, tebastanib, or MEDI3617), or derivative thereof, administered, or other drugs employed in combination, the mode of administration, and the like. An appropriate “effective” amount in any individual case may be determined by one of ordinary skill in the art using routine experimentation, based upon the information provided herein.

In certain embodiments, multiple therapeutically effective doses of compositions comprising pimagedine, atacicept, or an ANG-2 inhibitor (e.g., trebananib, tebastanib, or MEDI3617), and/or one or more other therapeutic agents, such as one or more other agents for treating proliferative diabetic retinopathy, such as, but not limited to corticosteroids such as, but not limited to, triamcinolone and fluocinolone acetonide; vascular endothelial growth factor inhibitors such as, but not limited to, bevacizumab, aflibercept, and ranibizumab; or other medications will be administered. The compositions comprising pimagedine, atacicept, or an ANG-2 inhibitor (e.g., trebananib, tebastanib, or MEDI3617) are typically, although not necessarily, administered intravitreally, e.g., by intravitreal injection or with an intravitreal implant (e.g., slow-release formulation), topically (e.g., eye drops), intraocularly, or locally. Additional modes of administration are also contemplated, such as oral, subcutaneous, intramuscular, intra-arterial, intravascular, pulmonary, intralesional, and so forth.

Preparations may also be suitable for local treatment. For example, compositions comprising pimagedine, atacicept, or an ANG-2 inhibitor (e.g., trebananib, tebastanib, or MEDI3617) may be administered locally to the eye by intravitreal injection or with an intravitreal implant. The particular preparation and appropriate method of administration can be chosen to target the pimagedine, atacicept, or an ANG-2 inhibitor (e.g., trebananib, tebastanib, or MEDI3617) to sites of neovascularization, retina damage, damaged or abnormal blood vessels, or macular edema in the eye. Local treatment may avoid some side effects of systemic therapy.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising pimagedine, atacicept, or an ANG-2 inhibitor (e.g., trebananib, tebastanib, or MEDI3617), and/or other agents may be administered using the same or different routes of administration in accordance with any medically acceptable method known in the art.

In another embodiment, the pharmaceutical compositions comprising pimagedine, atacicept, or an ANG-2 inhibitor (e.g., trebananib, tebastanib, or MEDI3617) and/or other agents are administered prophylactically, e.g., to prevent proliferative diabetic retinopathy. Such prophylactic uses will be of particular value for subjects who are at risk due to having diabetes (type I or type 1l) or a genetic predisposition to developing PDR.

In another embodiment, the pharmaceutical compositions comprising pimagedine, atacicept, or an ANG-2 inhibitor (e.g., trebananib, tebastanib, or MEDI3617), or a derivative thereof, and/or other agents for treating proliferative diabetic retinopathy, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions the pimagedine, atacicept, or ANG-2 inhibitor (e.g., trebananib, tebastanib, or MEDI3617) or a derivative thereof can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising pimagedine, atacicept, or an ANG-2 inhibitor (e.g., trebananib, tebastanib, or MEDI3617) or a derivative thereof will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising pimagedine, atacicept, or an ANG-2 inhibitor (e.g., trebananib, tebastanib, or MEDI3617) will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the pimagedine, atacicept, or an ANG-2 inhibitor (e.g., trebananib, tebastanib, or MEDI3617) or the derivative thereof and/or other agents administered, the magnitude of the effect desired, and the route of administration.

Pimagedine, atacicept, or an ANG-2 inhibitor (e.g., trebananib, tebastanib, or MEDI3617) or a derivative thereof (again, preferably provided as part of a pharmaceutical preparation) can be administered alone or in combination with one or more other therapeutic agents, such as other agents for treating proliferative diabetic retinopathy, including, but not limited to, corticosteroids (e.g., triamcinolone and fluocinolone acetonide) and vascular endothelial growth factor inhibitors (e.g., ranibizumab, aflibercept, and bevacizumab); dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

The pimagedine, atacicept, or an ANG-2 inhibitor (e.g., trebananib, tebastanib, or MEDI3617) or a derivative thereof can be administered prior to, concurrent with, or subsequent to other agents. If provided at the same time as other agents, pimagedine, atacicept, or an ANG-2 inhibitor (e.g., trebananib, tebastanib, or MEDI3617) or a derivative thereof can be provided in the same or in a different composition. Thus, pimagedine, atacicept, or an ANG-2 inhibitor (e.g., trebananib, tebastanib, or MEDI3617) or a derivative thereof and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising pimagedine, atacicept, or an ANG-2 inhibitor (e.g., trebananib, tebastanib, or MEDI3617) or a derivative thereof and a dose of a pharmaceutical composition comprising at least one other agent, such as another drug for treating proliferative diabetic retinopathy, which in combination comprise a therapeutically effective dose, according to a particular dosing regimen. Similarly, pimagedine, atacicept, or an ANG-2 inhibitor (e.g., trebananib, tebastanib, or MEDI3617) or a derivative thereof and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

Additionally, treatment with pimagedine, atacicept, or an ANG-2 inhibitor (e.g., trebananib, tebastanib, or MEDI3617) or a derivative thereof may be combined with any other medical treatment for proliferative diabetic retinopathy, such as, but not limited to, administering a corticosteroid such as, but not limited to, triamcinolone or fluocinolone acetonide; administering a vascular endothelial growth factor inhibitor such as, but not limited to, bevacizumab, aflibercept, or ranibizumab; performing laser surgery such as, but not limited to, photocoagulation or panretinal photocoagulation; or performing a vitrectomy; or any combination thereof.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-30 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of diagnosing and treating proliferative diabetic retinopathy (PDR) in a patient, the method comprising:
   a) obtaining a vitreous sample from an eye of the patient;
   b) measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the vitreous sample, wherein decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) compared to reference value ranges for a vitreous sample from a control subject, and increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) compared to reference value ranges for a vitreous sample from a control subject indicate that the patient has PDR; and
   c) treating the patient for the PDR, if the patient has a positive diagnosis for PDR.
2. The method of aspect 1, wherein the levels of expression of at least two, at least three, or at least four biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) are measured in the vitreous sample.
3. The method of aspect 1, wherein the levels of expression of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1 b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) are measured in the vitreous sample.
4. The method of any one of aspects 1 to 3, wherein said treating the patient for the PDR comprises administering a corticosteroid or a vascular endothelial growth factor (VEGF) inhibitor or performing laser surgery or a vitrectomy, or any combination thereof.
5. The method of aspect 4, wherein the corticosteroid is triamcinolone or fluocinolone acetonide.
6. The method of aspect 4, wherein the VEGF inhibitor is bevacizumab, aflibercept, or ranibizumab.
7. The method of aspect 4, wherein the laser surgery is photocoagulation or panretinal photocoagulation.
8. The method of any one of aspects 1 to 7, wherein said measuring the levels of expression comprises performing mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, fluorescence-activated cell sorting (FACS), or a Western Blot.
9. The method of aspect 8, wherein the ELISA is performed using a multiplex ELISA array.
10. A method of monitoring proliferative diabetic retinopathy (PDR) in a patient, the method comprising:
   a) obtaining a first vitreous sample from an eye of the patient at a first time point and a second vitreous sample from the eye of the subject later at a second time point;
   b) measuring one or more biomarkers in the first vitreous sample and the second vitreous sample, wherein the biomarkers are selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL); and
   c) analyzing the levels of expression of the one or more biomarkers in conjunction with respective reference value ranges for said biomarkers, wherein detection of decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and detection of increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening, and detection of increased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and decreased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL in the second vitreous sample compared to the first vitreous sample indicate that the patient is improving.
11. A method of monitoring efficacy of a treatment of a patient for proliferative diabetic retinopathy (PDR), the method comprising:
   a) obtaining a first vitreous sample from the patient before the patient undergoes the treatment and a second vitreous sample from the subject after the patient undergoes the treatment;
   b) measuring one or more biomarkers in the first vitreous sample and the second vitreous sample, wherein the biomarkers are selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL); and
   c) evaluating the efficacy of the treatment, wherein detection of decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and detection of increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening or not responding to the treatment, and detection of increased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1 BB) and decreased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL in the second vitreous sample compared to the first vitreous sample indicate that the patient is improving.
12. The method of aspect 11, further comprising altering the treatment if the patient is worsening or not responding to the treatment.
13. A kit comprising agents for detecting at least 3 biomarkers selected from the group consisting of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL).
14. The kit of aspect 13, wherein the kit comprises agents for detecting a biomarker panel comprising or consisting of all of the interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) biomarkers.
15. The kit of aspect 13 or 14, further comprising reagents for performing an immunoassay.
16. The kit of any one of aspects 13 to 15, wherein the kit comprises an antibody that specifically binds to interleukin 12 (IL-12p70), an antibody that specifically binds to thyroid-stimulating hormone (TSH), an antibody that specifically binds to interleukin-1 family member 6 (IL-1 F6), an antibody that specifically binds to fibroblast growth factor 6 (FGF-6), an antibody that specifically binds to bone morphogenetic protein 7 (BMP-7), an antibody that specifically binds to interleukin 1 beta (IL-1b), an antibody that specifically binds to follicle-stimulating hormone (FSH), an antibody that specifically binds to tafazzin (TAZ), an antibody that specifically binds to interleukin 34 (IL-34), an antibody that specifically binds to carcinoma antigen 15-3 (CA15-3), an antibody that specifically binds to albumin (ALB), an antibody that specifically binds to cadherin-6 (CDH6), an antibody that specifically binds to semaphorin 6D (SEMA6D), an antibody that specifically binds to tenascin R (TNR), an antibody that specifically binds to tumor necrosis factor receptor superfamily member 9 (4-1 BB), an antibody that specifically binds to vascular endothelial growth factor (VEGF), an antibody that specifically binds to galectin-2 (LGALS2), an antibody that specifically binds to transmembrane activator and CAML interactor (TACI), an antibody that specifically binds to monocyte chemoattractant protein 1 (MCP-1), an antibody that specifically binds to angiopoietin 2 (ANG-2), an antibody that specifically binds to interferon gamma-induced protein 10 (IP-10), an antibody that specifically binds to galectin-3 (LGALS3), an antibody that specifically binds to plasminogen activator inhibitor-1 (PAI-1), an antibody that specifically binds to C—C motif chemokine ligand 4 (MIP-1b), an antibody that specifically binds to cluster of differentiation 97 (CD97), an antibody that specifically binds to platelet-derived growth factor having 2 A subunits (PDGF-AA), an antibody that specifically binds to regenerating family member 4 (REG4), an antibody that specifically binds to nucleosome assembly protein 1 like 4 (NAP-2), an antibody that specifically binds to ferritin, matrix metallopeptidase 13 (MMP-13), an antibody that specifically binds to cluster of differentiation 99 (CD99), an antibody that specifically binds to B cell maturation antigen (BCMA), an antibody that specifically binds to cathepsin S (CTSS), an antibody that specifically binds to T cell immunoglobulin mucin 3 (TIM-3), midkine (MDK), an antibody that specifically binds to glycoxalase II, and an antibody that specifically binds to mannose-binding lectin (MBL).
17. The kit of any one of aspects 13 to 16, further comprising instructions for determining whether a subject has proliferative diabetic retinopathy (PDR).
18. A protein selected from the group consisting of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) for use as a biomarker in diagnosing proliferative diabetic retinopathy (PDR).
19. An in vitro method of diagnosing proliferative diabetic retinopathy (PDR), the method comprising:
   a) obtaining a vitreous sample from an eye of the patient; and
   b) measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1 BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the vitreous sample, wherein decreased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) compared to reference value ranges for a vitreous sample from a control subject, and increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) compared to reference value ranges for a vitreous sample from a control subject indicate that the patient has PDR.
20. A method of treating a subject for proliferative diabetic retinopathy, the method comprising administering a therapeutically effective amount of pimagedine, atacicept, or an ANG-2 inhibitor to the subject.
21. The method of aspect 20, wherein the ANG-2 inhibitor is trebananib, tebastanib, or MEDI3617.
22. The method of aspect 20 or 21, wherein the pimagedine, atacicept, or ANG-2 inhibitor is administered locally to the eye.
23. The method of aspect 22, wherein the pimagedine, atacicept, or ANG-2 inhibitor is administered locally to the retina.
24. The method of any one of aspects 20 to 23, wherein the pimagedine, atacicept, or ANG-2 inhibitor is administered intravitreally, intravenously, subcutaneously, or orally.
25. The method of any one of aspects 20 to 24, wherein multiple therapeutically effective doses of the pimagedine, atacicept, or ANG-2 inhibitor are administered to the subject.
26. The method of aspect 25, wherein the pimagedine, atacicept, or ANG-2 inhibitor are administered daily or intermittently.
27. A composition comprising pimagedine, atacicept, or an ANG-2 inhibitor for use in a method of treating proliferative diabetic retinopathy.
28. The composition of aspect 27, wherein the ANG-2 inhibitor is trebananib, tebastanib, or MEDI3617.
29. A composition comprising pimagedine, atacicept, or an ANG-2 inhibitor for use as a medicament for the treatment of proliferative diabetic retinopathy.
30. The composition of aspect 29, wherein the ANG-2 inhibitor is trebananib, tebastanib, or MEDI3617.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Methods for Diagnosing and Treating Ischemic Eye Disease

### INTRODUCTION

Advances in proteomics and genomics are leading to the development of more precise interventions for vitreoretinal diseases like PDR. Proteomic analysis is becoming an attractive and powerful method for characterizing the molecular profiles of diseased tissues and identifying targets of existing drugs (i.e. drug repositioning).⁶⁻⁸ Furthermore, proteomics can identify dynamic changes in protein expression in response that correlate with disease timing, severity, and response to therapy. There have been several proteomic studies on human PDR vitreous looking at the differential expression of anywhere from 25 to 415 proteins (Table 1). However, a majority of these studies utilized “shotgun” mass spectrometry (MS)-based approaches, while few employed quantitative and targeted proteomic platforms (e.g., multiplex ELISA) with a maximum number of 27 proteins. While highly informative, there is often an intrinsic gap between shotgun discovery-based approaches and clinically applicable results. Although MS-based experiments promote the identification of thousands of proteins in a single screen, the digested and fragmented peptides are usually represented relative to the abundance of each protein in a biological sample. Thus, more abundant proteins are often identified, and less abundant but clinically relevant protein biomarkers go undetected. Therefore, the implementation of more direct, hypothesis driven platforms can streamline the biomarker discovery process by measuring more viable, validated, and clinically translatable analytes for drug repositioning.⁹

VEGF continues to be a common target of diabetic retinopathy therapy as high levels have been detected in the vitreous of patients with PDR.¹⁰ It was not initially identified through proteomic studies though findings would have strongly supported its targeting.^{11, 12} While anti-VEGF therapies have proven successful, they carry safety issues at the ocular and systemic levels.¹³⁻¹⁸ Furthermore, 30% of patients with PDR fail to respond to treatment and those who do respond require multiple injections.^{16, 19, 20} Our group has used proteomics of human vitreous fluid to understand that molecules driving disease and successfully identified existing drugs for repositioning.²¹ A similar approach may be beneficial in uncovering new treatment options for PDR. Here, we used a high-throughput antibody array to detect and quantify 1,000 different protein biomarkers in vitreous biopsies from PDR patients.

### Methods

Study approval—The study was approved by the Stanford University Institutional Review Board and adhered to the tenets set forth in the Declaration of Helsinki. Subjects underwent eye exams that included slit-lamp examination, dilated retinal bio-microscopy and indirect ophthalmoscopy.

Sample collection—Pars plana vitrectomy (PPV) was performed using a single-step transconjunctival 25-gauge trocar cannular system (Alcon Laboratories Inc, Fort Worth, TX), and an undiluted 0.5-cc sample of the vitreous was manually aspirated into a 3-cc syringe. Vitreous samples were immediately frozen and stored at −80° C., as previously described.²²

Proteomic analysis—Targeted quantitative proteomics was performed on liquid vitreous biopsies using a multiplex ELISA array that concurrently detected and processed 1,000 human proteins. Multiplex ELISA combines the high specificity and sensitivity of an ELISA with the high throughput of a glass chip-based array. Only 50 microliters of sample are required for quantification of up to forty proteins, making it 80 times more efficient than a standard ELISA. Vitreous cytokine signaling proteins were measured using the Human Kiloplex Array Q1 (RayBio, Norcross, GA) per the manufacturers protocol. First, the array chips were incubated with sample diluents for 30 minutes at room temperature to act as a block. Diluted vitreous (four technical replicates per sample; 1.5 mL total) was added to the wells of the array and incubated overnight at 4° C. A standard protein dilution was added to the wells of the array to determine protein concentrations. For signal detection, 80 μL of Cy3-streptavidin was added to each well, rinsed and visualized by laser scanner. The RayBio® Analysis Tool (RayBio®, Norcross, GA) was used for protein classification. Final protein concentrations (in pg/mL) were corrected for sample dilution. Biopsies were analyzed using this membrane-based antibody array, to identify any abnormally expressed proteins, and determine a PDR protein signature.

Statistical and bioinformatic analysis—Results from the separate datasets were saved in Excel as .txt format and were uploaded into the Partek Genomics Suite 6.5 software package. The data was normalized to log base 2 and compared using 1-way ANOVA analysis. All proteins with non-significant (p>0.05) changes were eliminated from the table. The significant values were heat-mapped using the cluster based on significant genes' visualization function with the standardization option chosen. Principal component analysis was performed using Qlucore Omics Explorer 3.2 software.

Pathway and network analysis—Since the multiplex ELISA does not represent the entire vitreous proteome, we used an overrepresentation analysis (ORA)-based method with the 1,000 proteins on the array as the reference protein list.²³⁻²⁵ Enriched pathways were detected by searching the PANTHER²⁶ and KEGG²⁷ databases using the WebGestalt toolkit.²⁸ Pathway analysis was performed using an ORA method followed by Benjamini-Hochberg multiple test adjustments.²⁴ The full list of 1,000 proteins represented on the Human Kiloplex Array Q1 (raybiotech.com/human-kiloplex/) was used as the reference protein list. Gene ontology analysis was also performed in PANTHER.²⁹ Pie charts were created for the visualization of GO distributions within the list of proteins under the Batch ID search menu. Pie charts were created for each GO term category including biological process, molecular function, and cellular compartment. Protein networks and interactomes were constructed using STRING and visualized in Cytoscape as previously described.^{22,30-33}

Drug repositioning—We searched the DrugBank database for drugs (both investigational and approved) targeting the identified proteins in our proteomics screen. DrugBank is a detailed database that contains comprehensive drug target and drug action information.³⁴

### Case Series

Undiluted vitreous biopsies were collected from both control subjects (n=6) undergoing PPV to remove an idiopathic macular hole (IMH), and test subjects (n=6) with PDR (Table 2). The median age was higher for controls than cases (median 68, interquartile range (IQR) 9.5 versus median 43, IQR 9). Most samples were from male patients with 4/6 male cases and 5/6 male controls. More cases than controls were phakic (5/6 cases, 2/6 controls). Four cases were undergoing PPV for non-clearing vitreous hemorrhage, while two were undergoing surgery for TRDs, one of which was a combined TRD, RRD. In terms of systemic diabetes diagnosis, 3/6 were insulin dependent type I diabetics (IDDM) while 3/6 were non-insulin dependent (NIDDM) type II diabetics. The controls all had epiretinal membranes but had a wide array of concurrent pathology including mild branch retinal vein occlusion, peripapillary choroidal neovascular membrane (CNVM), retinoschisis, choroidal nevus, CME, and RRD status post buckle repair. One control had NIDDM, Type II without retinopathy while another had mild NPDR.

### Results

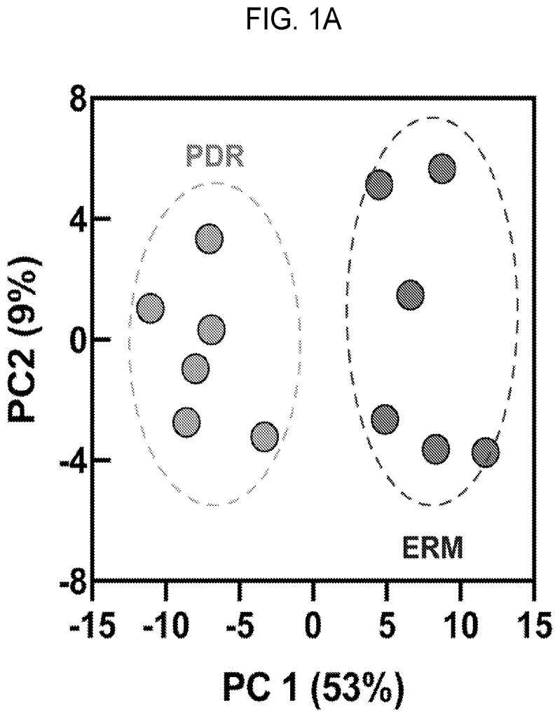

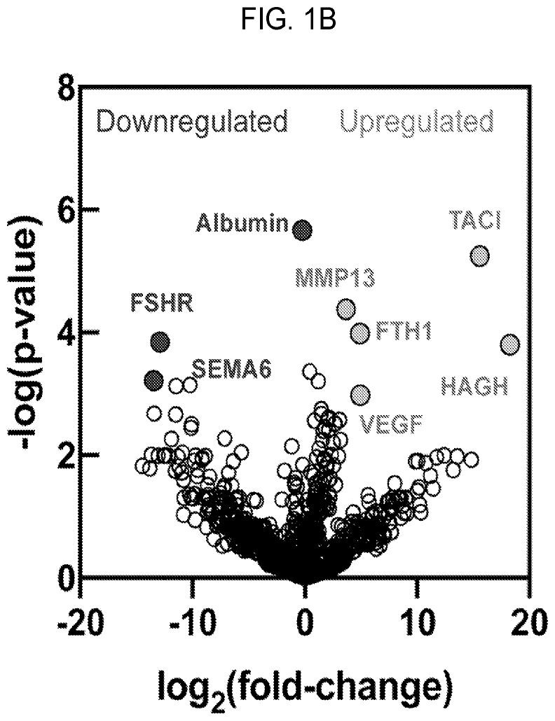

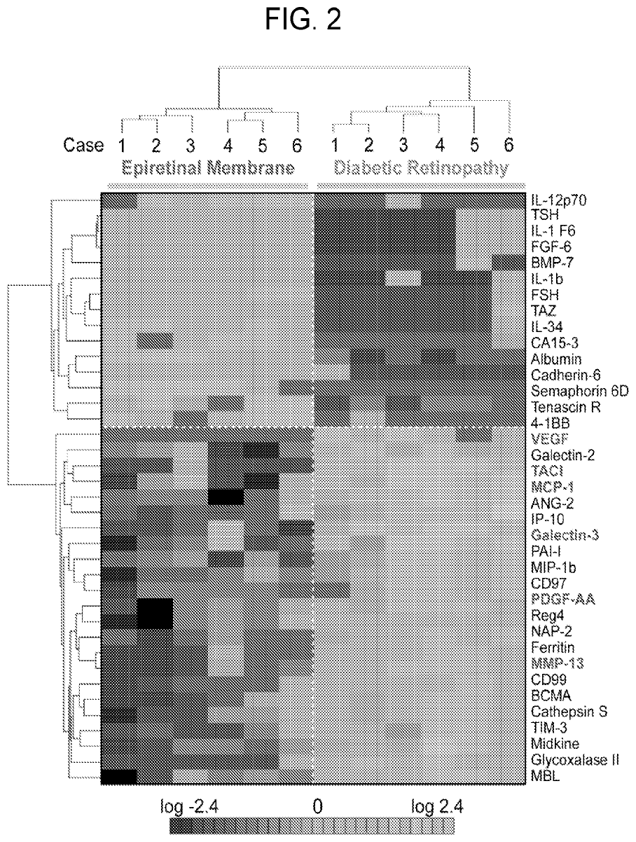

Targeted proteomic analysis reveals novel therapeutic targets for proliferative diabetic retinopathy—Expression of one-thousand proteins was measured in the vitreous samples. The score plot of PC1 and PC2 showed separation between the 6 diabetic retinopathy cases and 6 controls based on differentially expressed proteins that were significantly different between the two groups (false-discovery rate=0.017, adjusted p-value=0.00004; FIG. 1). This result gave confidence that vitreous protein signatures that distinguish patients with diabetic retinopathy from controls could be determined. Protein expression was further analyzed using 1-way ANOVA and hierarchical heatmap clustering. We identified a total of 37 unique proteins in PDR vitreous and 79 unique proteins in control samples. A total of 125 proteins were differentially expressed among control and PDR samples (66 upregulated proteins and 60 downregulated proteins; p<0.05). Upon further restricting the sample to p<0.01, a total of 37 proteins were differentially expressed among control and PDR samples (27 upregulated proteins and 15 downregulated proteins; FIG. 2). With the exception of VEGF and ANG-2, none of the significantly upregulated proteins have been previously identified in the PDR vitreous proteome (Table 1; p<0.01).³⁵

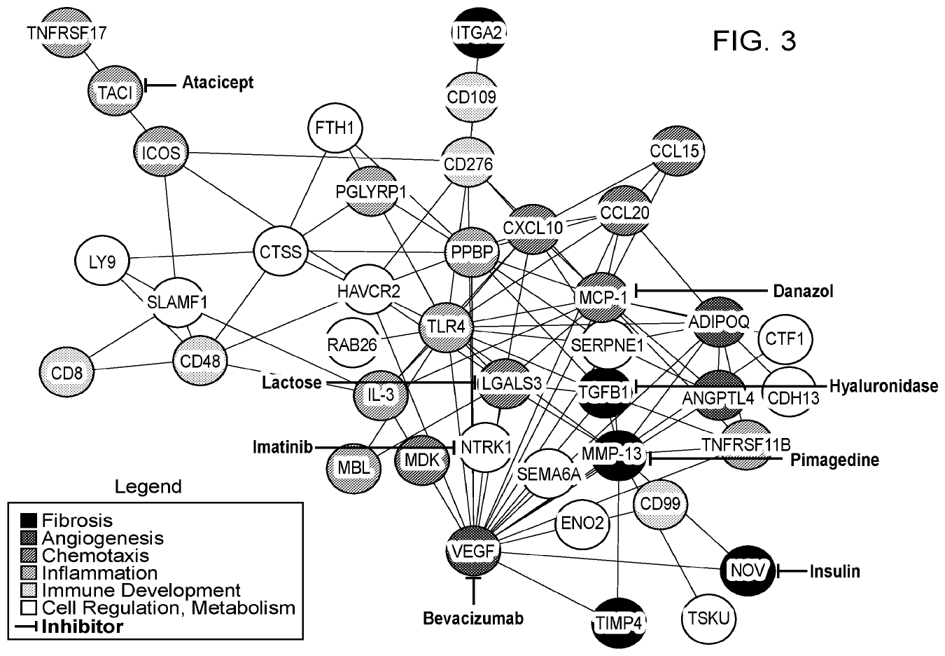

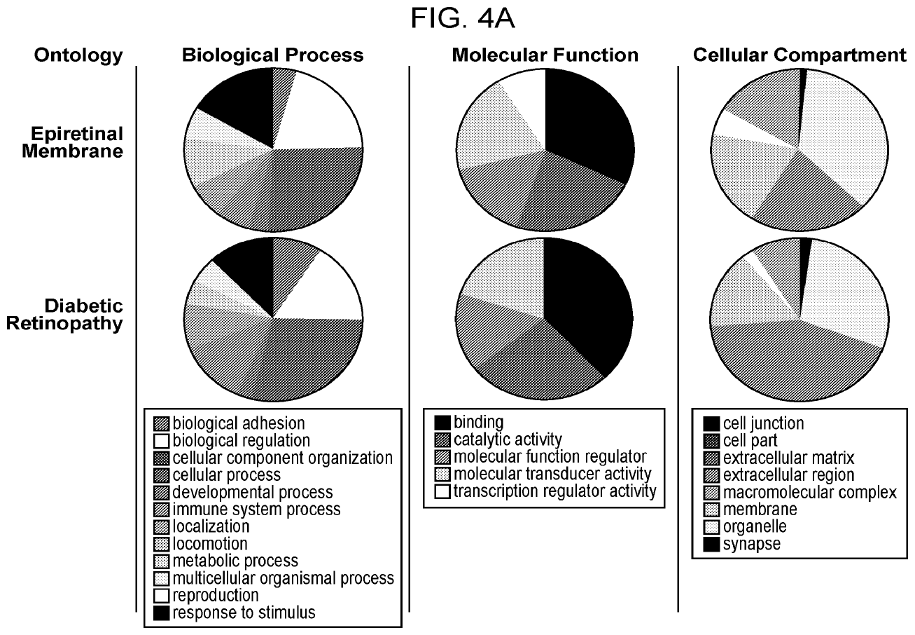

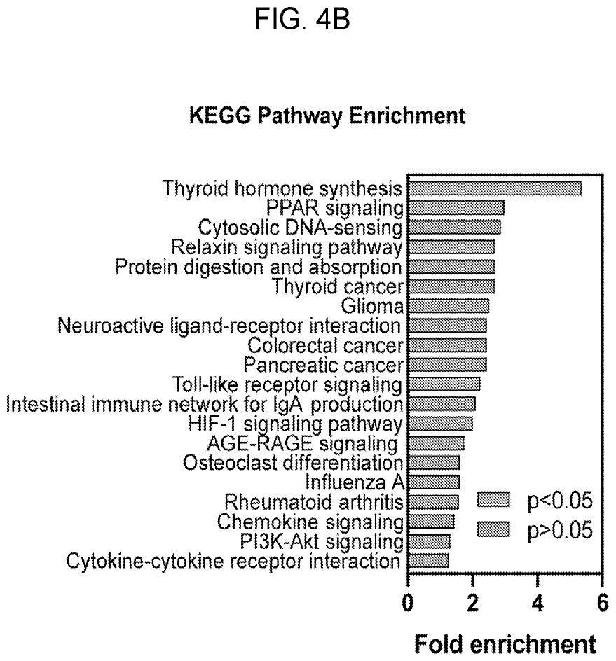

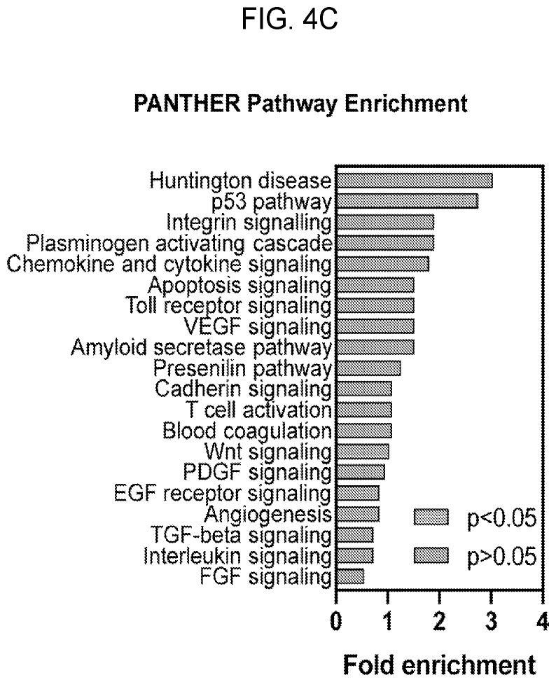

Pathway representation—Molecular pathway analysis was used to identify functionally linked proteins in PDR vitreous samples. When analyzing the differentially expressed proteins from both PDR and control samples, the most represented pathways included: fibrosis, inflammation, angiogenesis, chemotaxis, cell regulation & metabolism, and immune development (FIG. 4). We constructed a network of protein-protein interactions among the upregulated proteins in PDR vitreous (FIG. 3). Several components of the fibrosis pathway were identified, including MMP-13, NOV, TGF-β1, TIMP4 and ITGA2. Matrix metalloproteinase 13 (MMP-13, collagenase 3) is a member of the peptidase M10 family of matrix metalloproteinases. It degrades collagenous extracellular matrix (ECM) and is associated with both normal physiological processes such as embryonic development and tissue remodeling as well as pathological processes such as osteoarthritis, atherosclerosis, cancer and fibrosis.

Among the upregulated proteins was nephroblastoma overexpressed (NOV, also known as CCN3), a member of the CCN family of secreted ECM associated signaling proteins known to play a critical role in obesity and diabetes.³⁶ The CCN family of growth factors is critical in fibrosis but also angiogenesis, inflammation, cellular growth, differentiation, and adhesion and movement in wound repair. CCN3/NOV has previously been reported as potentially protective against diabetes end-organ complications.³⁶ Here, however, we found NOV upregulated in the vitreous of patients with PDR. We also identified elevated transforming growth factor β (TGF-β) levels. TGF-β is a secreted signaling molecule that controls a wide range of biological processes.³⁷ In the retina, TGF-β is primarily expressed by RPE cells and pericytes,^{38, 39} and it has roles regulating neuronal differentiation and survival,^{40, 41} as well as maintenance of the retinal microvasculature.⁴² It has previously been reported that TGF-β levels in vitreous samples of patients with active PDR are reduced by 80% relative to controls, as measured by ELISA.⁴³ However, the role of TGF-β in PDR remains controversial, as there is evidence to support both pro-angiogenic and anti-angiogenic roles.

We identified several elevated inflammatory and chemotactic factors: TACI, TLR4, MBL, IL-3, PPBP, ICOS, TNFRSF11B, and TNFRSF17. Amongst these, TACI is a notable protein that has not previously been reported in the vitreous of diabetics. Transmembrane activator and CAML (calcium-modulating cyclophilin ligand) interactor (TACI) is a transmembrane protein receptor found predominantly on the surface of B cells, but also on activated T cells. It is shared by two members of the TNF (tumor necrosis factor) superfamily, APRIL (TNFSF13) and BAFF (the B-cell activation factor of the TNF family).⁴⁴ TACI binding leads APRIL and BAFF to activate macrophages and initiate inductors of mediators of pro-inflammatory cytokines and matrix degrading enzymes.^{45,46} It is a protein of interest here because it was highly upregulated in patients with PDR and only present in the vitreous humor of one control patient. Within the chemotaxis pathway, MCP-1 functions to attract monocytes and basophils, but not neutrophils or eosinophils.^{47,48} While it is produced by a wide variety of cell types, it is chiefly secreted by monocytes/macrophages. MCP-1 is involved in a number of inflammatory and autoimmune diseases, cancers, vascular diseases, and ocular diseases (e.g. proliferative vitreoretinopathy, PVR).⁴⁹ Circulating MCP-1 is significantly increased in diabetes. MCP-1 has been found to modulate microglia and neuroprotection.^{50, 51}

The angiogenesis pathway is critical in the pathogenesis of PDR, and we identified elevated levels of members of the angiogenesis pathway, including VEGF, ANG-2, adiponectin (ADIPOQ), and angiopoietin-related protein 4 (ANGPTL4). ANG-2 is a broadly expressed, secreted glycoprotein belonging to the four-member angiopoietin family in humans. It is involved in promoting angiogenesis and modulating inflammation, serving as an antagonist of ANG-1 and TIE-2.⁵² ANG-2 cooperates with VEGF to induce vascular sprouting, but induces endothelial cell apoptosis in the absence of VEGF. Additionally, ANG-2 is generally considered a pro-inflammatory factor. In the context of diabetes, circulating ANG-2 levels are known to be elevated, but the consequences of this elevation remain unknown.⁵³ In the eye, increased levels of ANG-2 have been observed previously in the vitreous of patients with PDR.³⁵

### Drug Repositioning

It is possible that PDR patients might benefit from already available therapeutics or natural compounds. We used the DrugBank database (go.drugbank.com/), a source launched in 2006 that contains drug-protein interactions and has more than a hundred information fields for each drug. Unique PDR proteins were run in DrugBank as protein targets to identify drug-protein relationships. From the 38 unique proteins in PDR vitreous, 8 drugs known to inhibit these proteins were identified. Based on the significance of MMP-13 in cleaving the most abundant form of collagen in the vitreous, we explored existing pharmacologic treatments that could target MMP-13. MMP-13 is down regulated by the drug Pimagedine, or aminoguanidine, an inducible nitric oxide synthase inhibitor. Pimagedine was initially marketed for the treatment of diabetic nephropathy,^{54,55} but the systemic form has been shown to prevent retinopathy in rats and dogs.⁵⁶ It was recently injected intravitreally in diabetic rabbits and found to be safe in human equivalent doses of 2.7 mg and 1 mg.⁵⁷ Of note no clinical trials are currently registered. TACI similarly is the target of a known drug—Atacicept—a recombinant fusion protein with the extracellular ligand-binding portion of the TACI receptor fused to the Fc of human IgG1. It binds to B-lymphocyte stimulator (BLyS) and APRIL and thus interferes with B-cell signal transduction otherwise induced by these ligands.⁵⁸ Its administration has been shown to decrease levels of naïve B cells, plasma cells and serum immunoglobulin.^{59,60} It was found to be safe and efficacious in a Phase IIb study of patients with autoantibody-positive lupus.⁶¹ Conversely, a Phase II randomized, double-blind, placebo-controlled trial on 34 patients with unilateral optic neuritis as a clinically isolated syndrome was terminated early as a higher proportion of atacicept-treated patients converted to relapsing-remitting MS as compared with placebo-treated patients.⁶² Though, of note, the same patients experienced less axonal loss after an optic neuritis event.⁶² ANG-2 is also the target of known drugs (i.e. Trebananib, Rebastanib, and MEDI3617) that are currently being evaluated in clinical trials for recurrent or metastatic breast and renal cell carcinomas.⁶³ Either Pimagedine, Atacicept, or an ANG-2 inhibitor could be evaluated for drug repositioning for PDR.

### DISCUSSION

There were an estimated 451 million people living with diabetes in 2017 with an estimated expenditure of approximately 850 billion US dollars and the prevalence of both diabetes and diabetic retinopathy are expected to continue to rise.⁶⁴ A major issue in the management of PDR is the use of therapies and drugs with unfavorable side effect profiles. The mainstays of treatment are panretinal photocoagulation, focal laser, and intravitreal anti-VEGF therapy.⁶⁵ Disadvantages of laser include the potential for permanent visual field loss and permanent alteration to the physiologic structure of the eye. Newer anti-VEGF therapy may have toxic effects when used long-term and may also have systemic side effects on the kidneys and heart.¹⁸ Recurrence is common at 12 weeks after treatment, necessitating frequent repeat therapy.^{19, 20, 66} Additionally, some patients do not respond to either therapy. Using our proteomics platform, we have identified novel therapeutic targets for PDR that point to new medications that may supplement a therapy plan.

In the current study, we performed a targeted, quantitative proteomics analysis of PDR vitreous. As compared to control vitreous, PDR vitreous was found to have higher expression of certain proteins including MMP-13, TACI, ANG-2, MCP-1, TGF-β, and VEGF. MMP-13 is a pro-fibrotic and pro-angiogenic enzyme. It has been shown to promote VEGF-A secretion from fibroblasts and endothelial cells, encouraging researchers to consider that MMP-13 may directly and indirectly promote tumor angiogenesis.⁶⁷ Murine studies add to the evidence that MMP-13 is critical in both fibrosis and angiogenesis as MMP-13 knockout mice have delayed angiogenic responses in development. In a murine experimental retinal detachment, MMP-13 was significantly elevated. Immunohistochemistry revealed moderate to strong MMP-13 levels in subretinal macrophages.⁶⁸ In the eye, MMP-13 produced by bone marrow-derived cells has been associated with both CNVM and retinal detachments.^{68,69} While MMP-1 and MMP-8 primarily cleave collagen type II in human vitreous, MMP-13 is also capable of cleaving collagen type II and may be invoked to do so at a higher rate in a pathologic eye.⁷⁰ In a prior study of 32 patients with PDR and 24 nondiabetic controls, MMPs were studied by ELISA, immunoblot and zymography analysis.⁷¹ Significant elevations in expression of MMP-1, MMP-7, and MMP-9 were found and elevations of MMP-1 and MMP-9 correlated with VEGF elevations. MMP-8 and MMP-13 were not detected despite being on the utilized ELISA array. Possible explanations for the lack of MMP-13 detection in prior studies include differences in patient disease stage (i.e. early vs. late stage PDR), antibody quality, and sample processing. Nevertheless, the detection of elevated MMP-13 in PDR vitreous is both novel and fitting given its ability to process type II collagen.

Limitations of this study include that the multiplex ELISA array does not measure complement. Complement is believed to be play in important role in the vitreous.⁷² However, it largely acts as an acute phase reactant with inflammation without playing a specific mechanistic role. Additionally, complement may be a poor target of drug repurposing as inhibiting complement may increase the risk of intravitreal injection-associated endophthalmitis.⁷³ In any pilot study to establish a discovery data set for future targeted validation, a limitation will be small sample size and heterogenicity in the patient population presented. Additionally, the study is limited by the fact that the case group was significantly younger than the control group with a mean age of 43 versus 70. We know that the macromolecular composition and viscosity of vitreous is influenced by age, the state of the lens, and the presence of vitreous and retinal pathology. Therefore, older patients might be expected to have age related changes that may have affected variances in protein composition. In the future, we will need prospective validation of a smaller array through analysis of significantly regulated and clinically relevant proteins on a larger cohort of cases and controls. Our pilot study may also help to prioritize drugs for testing in pre-clinical PDR models. Our recently developed a murine model (i.e. Chx10-cre; Vhl^{flox/flox} mouse) that recapitulates the key vasculature defects of human PDR.⁷⁴ Development of novel intravitreal protease inhibitors may provide new treatments for PDR.

### REFERENCES

1. Ding J, Wong T Y. Current epidemiology of diabetic retinopathy and diabetic macular edema. Curr Diab Rep. 2012; 12(4):346-354.
2. Zheng Y, He M, Congdon N. The worldwide epidemic of diabetic retinopathy. Indian J Ophthalmol. 2012; 60(5):428-431.
3. Wert K J, Velez G, Cross M R, et al. Extracellular superoxide dismutase (SOD3) regulates oxidative stress at the vitreoretinal interface. Free Radic Biol Med. 2018; 124:408-419.
4. Nita M, Grzybowski A. The Role of the Reactive Oxygen Species and Oxidative Stress in the Pathomechanism of the Age-Related Ocular Diseases and Other Pathologies of the Anterior and Posterior Eye Segments in Adults. Oxid Med Cell Longev. 2016; 2016:3164734.
5. Frank R N. Diabetic retinopathy. N Engl J Med. 2004; 350(1):48-58.
6. Velez G, Roybal C N, Colgan D, Tsang S H, Bassuk A G, Mahajan V B. Precision Medicine: Personalized Proteomics for the Diagnosis and Treatment of Idiopathic Inflammatory Disease. JAMA Ophthalmol. 2016; 134(4):444-448.
7. Velez G, Tang P H, Cabral T, et al. Personalized Proteomics for Precision Health: Identifying Biomarkers of Vitreoretinal Disease. Transl Vis Sci Technol. 2018; 7(5):12.
8. Velez G, Machlab D A, Tang P H, et al. Proteomic analysis of the human retina reveals region-specific susceptibilities to metabolic- and oxidative stress-related diseases. PLoS One. 2018; 13(2):e0193250.
9. Faria S S, Morris C F, Silva A R, et al. A Timely Shift from Shotgun to Targeted Proteomics and How It Can Be Groundbreaking for Cancer Research. Front Oncol. 2017; 7:13.
10. Clermont A C, Aiello L P, Mori F, Aiello L M, Bursell S E. Vascular endothelial growth factor and severity of nonproliferative diabetic retinopathy mediate retinal hemodynamics in vivo: a potential role for vascular endothelial growth factor in the progression of nonproliferative diabetic retinopathy. Am J Ophthalmol. 1997; 124(4):433-446.
11. Fong A H, Lai T Y. Long-term effectiveness of ranibizumab for age-related macular degeneration and diabetic macular edema. Clin Interv Aging. 2013; 8:467-483.
12. Nguyen Q D, Schachar R A, Nduaka C I, et al. Dose-ranging evaluation of intravitreal siRNA PF-04523655 for diabetic macular edema (the DEGAS study). Invest Ophthalmol Vis Sci. 2012; 53(12):7666-7674.
13. Cheung N, Wong I Y, Wong T Y. Ocular anti-VEGF therapy for diabetic retinopathy: overview of clinical efficacy and evolving applications. Diabetes Care. 2014; 37(4):900-905.
14. Georgalas I, Papaconstantinou D, Papadopoulos K, Pagoulatos D, Karagiannis D, Koutsandrea C. Renal injury following intravitreal anti-VEGF administration in diabetic patients with proliferative diabetic retinopathy and chronic kidney disease—a possible side effect? Curr Drug Saf. 2014; 9(2):156-158.
15. Lains I, Figueira J, Santos A R, et al. Choroidal thickness in diabetic retinopathy: the influence of antiangiogenic therapy. Retina. 2014; 34(6):1199-1207.
16. Arevalo J F, Wu L, Sanchez J G, et al. Intravitreal bevacizumab (Avastin) for proliferative diabetic retinopathy: 6-months follow-up. Eye (Lond). 2009; 23(1):117-123.
17. Moradian S, Ahmadieh H, Malihi M, Soheilian M, Dehghan M H, Azarmina M. Intravitreal bevacizumab in active progressive proliferative diabetic retinopathy. Graefes Arch Clin Exp Ophthalmol. 2008; 246(12):1699-1705.
18. Simo R, Hernandez C. Intravitreous anti-VEGF for diabetic retinopathy: hopes and fears for a new therapeutic strategy. Diabetologia. 2008; 51(9):1574-1580.
19. Mirshahi A, Roohipoor R, Lashay A, Mohammadi S F, Abdoallahi A, Faghihi H. Bevacizumab-augmented retinal laser photocoagulation in proliferative diabetic retinopathy: a randomized double-masked clinical trial. Eur J Ophthalmol. 2008; 18(2):263-269.
20. Schmidinger G, Maar N, Bolz M, Scholda C, Schmidt-Erfurth U. Repeated intravitreal bevacizumab (Avastin(®)) treatment of persistent new vessels in proliferative diabetic retinopathy after complete panretinal photocoagulation. Acta Ophthalmol. 2011; 89(1):76-81.
21. Velez G, Bassuk A G, Colgan D, Tsang S H, Mahajan V B. Therapeutic drug repositioning using personalized proteomics of liquid biopsies. JCI Insight. 2017; 2(24).
22. Skeie J M, Tsang S H, Zande R V, et al. A biorepository for ophthalmic surgical specimens. Proteomics Clin Appl. 2014; 8(3-4):209-217.
23. Zhang B, Kirov S, Snoddy J. WebGestalt: an integrated system for exploring gene sets in various biological contexts. Nucleic Acids Res. 2005; 33(Web Server issue):W741-748.
24. Garcia-Campos M A, Espinal-Enriquez J, Hernandez-Lemus E. Pathway Analysis: State of the Art. Front Physiol. 2015; 6:383.
25. Kramer A, Green J, Pollard J, Jr., Tugendreich S. Causal analysis approaches in Ingenuity Pathway Analysis. Bioinformatics. 2014; 30(4):523-530.
26. Mi H, Thomas P. PANTHER pathway: an ontology-based pathway database coupled with data analysis tools. Methods Mol Biol. 2009; 563:123-140.
27. Kanehisa M, Goto S. KEGG: kyoto encyclopedia of genes and genomes. Nucleic Acids Res. 2000; 28(1):27-30.
28. Liao Y, Wang J, Jaehnig E J, Shi Z, Zhang B. WebGestalt 2019: gene set analysis toolkit with revamped Uls and APIs. Nucleic Acids Res. 2019; 47(W1):W199-W205.
29. Mi H, Muruganujan A, Casagrande J T, Thomas P D. Large-scale gene function analysis with the PANTHER classification system. Nat Protoc. 2013; 8(8):1551-1566.
30. Szklarczyk D, Franceschini A, Wyder S, et al. STRING v10: protein-protein interaction networks, integrated over the tree of life. Nucleic Acids Res. 2015; 43(Database issue):D447-452.
31. Skeie J M, Mahajan V B. Proteomic landscape of the human choroid-retinal pigment epithelial complex. JAMA Ophthalmol. 2014; 132(11):1271-1281.
32. Skeie J M, Roybal C N, Mahajan V B. Proteomic insight into the molecular function of the vitreous. PLoS One. 2015; 10(5):e0127567.
33. Skeie J M, Mahajan V B. Proteomic interactions in the mouse vitreous-retina complex. PLoS One. 2013; 8(11):e82140.
34. Wishart D S, Knox C, Guo A C, et al. DrugBank: a knowledgebase for drugs, drug actions and drug targets. Nucleic Acids Res. 2008; 36(Database issue):D901-906.
35. Watanabe D, Suzuma K, Suzuma I, et al. Vitreous levels of angiopoietin 2 and vascular endothelial growth factor in patients with proliferative diabetic retinopathy. Am J Ophthalmol. 2005; 139(3):476-481.
36. Twigg S M. Regulation and bioactivity of the CCN family of genes and proteins in obesity and diabetes. J Cell Commun Signal. 2018; 12(1):359-368.
37. Seuntjens E, Umans L, Zwijsen A, Sampaolesi M, Verfaillie C M, Huylebroeck D. Transforming Growth Factor type beta and Smad family signaling in stem cell function. Cytokine Growth Factor Rev. 2009; 20(5-6):449-458.
38. Eichler W, Yafai Y, Wiedemann P, Reichenbach A. Angiogenesis-related factors derived from retinal glial (Muller) cells in hypoxia. Neuroreport. 2004; 15(10):1633-1637.
39. Katsura M K, Mishima H K, Minamoto A, Ishibashi F, Yamashita H. Growth regulation of bovine retinal pericytes by transforming growth factor-beta2 and plasmin. Curr Eye Res. 2000; 20(3):166-172.
40. Braunger B M, Pielmeier S, Demmer C, et al. TGF-beta signaling protects retinal neurons from programmed cell death during the development of the mammalian eye. J Neurosci. 2013; 33(35):14246-14258.
41. Walshe T E, Leach L L, D′Amore P A. TGF-beta signaling is required for maintenance of retinal ganglion cell differentiation and survival. Neuroscience. 2011; 189:123-131.
42. Walshe T E, Saint-Geniez M, Maharaj A S, Sekiyama E, Maldonado A E, D'Amore P A. TGF-beta is required for vascular barrier function, endothelial survival and homeostasis of the adult microvasculature. PLoS One. 2009; 4(4):e5149.
43. Spranger J, Meyer-Schwickerath R, Klein M, Schatz H, Pfeiffer A. Deficient activation and different expression of transforming growth factor-beta isoforms in active proliferative diabetic retinopathy and neovascular eye disease. Exp Clin Endocrinol Diabetes. 1999; 107(1):21-28.
44. Zhang Y, Kent J W, Jr., Olivier M, et al. QTL-based association analyses reveal novel genes influencing pleiotropy of metabolic syndrome (MetS). Obesity (Silver Spring). 2013; 21(10):2099-2111.
45. Lee S M, Kim E J, Suk K, Lee W H. BAFF and APRIL induce inflammatory activation of THP-1 cells through interaction with their conventional receptors and activation of MAPK and NF-kappaB. Inflamm Res. 2011; 60(9):807-815.
46. Lee S M, Kim W J, Suk K, Lee W H. Cell to Cell Interaction Can Activate Membrane-bound APRIL Which Are Expressed on Inflammatory Macrophages. Immune Netw. 2010; 10(5):173-180.
47. Deshmane S L, Kremlev S, Amini S, Sawaya B E. Monocyte chemoattractant protein-1 (MCP-1): an overview. J Interferon Cytokine Res. 2009; 29(6):313-326.
48. Panee J. Monocyte Chemoattractant Protein 1 (MCP-1) in obesity and diabetes. Cytokine. 2012; 60(1):1-12.
49. Roybal C N, Velez G, Toral M A, Tsang S H, Bassuk A G, Mahajan V B. Personalized Proteomics in Proliferative Vitreoretinopathy Implicate Hematopoietic Cell Recruitment and mTOR as a Therapeutic Target. Am J Ophthalmol. 2018; 186:152-163.
50. Nakazawa T, Hisatomi T, Nakazawa C, et al. Monocyte chemoattractant protein 1 mediates retinal detachment-induced photoreceptor apoptosis. Proc Natl Acad Sci USA. 2007; 104(7):2425-2430.
51. Chiu K, Yeung S C, So K F, Chang R C. Modulation of morphological changes of microglia and neuroprotection by monocyte chemoattractant protein-1 in experimental glaucoma. Cell Mol Immunol. 2010; 7(1):61-68.
52. Scholz A, Plate K H, Reiss Y. Angiopoietin-2: a multifaceted cytokine that functions in both angiogenesis and inflammation. Ann NY Acad Sci. 2015; 1347:45-51.
53. Rasul S, Reiter M H, Ilhan A, Lampichler K, Wagner L, Kautzky-Willer A. Circulating angiopoietin-2 and soluble Tie-2 in type 2 diabetes mellitus: a cross-sectional study. Cardiovasc Diabetol. 2011; 10:55.
54. Abdel-Rahman E, Bolton W K. Pimagedine: a novel therapy for diabetic nephropathy. Expert Opin Investig Drugs. 2002; 11(4):565-574.
55. Bolton W K, Cattran D C, Williams M E, et al. Randomized trial of an inhibitor of formation of advanced glycation end products in diabetic nephropathy. Am J Nephrol. 2004; 24(1):32-40.
56. Kim J, Kim C S, Sohn E, et al. Aminoguanidine protects against apoptosis of retinal ganglion cells in Zucker diabetic fatty rats. Eur Rev Med Pharmacol Sci. 2014; 18(11):1573-1578.
57. Carr B C, Emigh C E, Bennett L D, Pansick A D, Birch D G, Nguyen C. TOWARDS A TREATMENT FOR DIABETIC RETINOPATHY: Intravitreal Toxicity and Preclinical Safety Evaluation of Inducible Nitric Oxide Synthase Inhibitors. Retina. 2017; 37(1):22-31.
58. Dillon S R, Harder B, Lewis K B, et al. B-lymphocyte stimulator/a proliferation-inducing ligand heterotrimers are elevated in the sera of patients with autoimmune disease and are neutralized by atacicept and B-cell maturation antigen-immunoglobulin. Arthritis Res Ther. 2010; 12(2):R48.
59. Genovese M C, Kinnman N, de La Bourdonnaye G, Pena Rossi C, Tak P P. Atacicept in patients with rheumatoid arthritis and an inadequate response to tumor necrosis factor antagonist therapy: results of a phase II, randomized, placebo-controlled, dose-finding trial. Arthritis Rheum. 2011; 63(7):1793-1803.
60. Isenberg D, Gordon C, Licu D, Copt S, Rossi C P, Wofsy D. Efficacy and safety of atacicept for prevention of flares in patients with moderate-to-severe systemic lupus erythematosus (SLE): 52-week data (APRIL-SLE randomised trial). Ann Rheum Dis. 2015; 74(11):2006-2015.
61. Merrill J T, Wallace D J, Wax S, et al. Efficacy and Safety of Atacicept in Patients With Systemic Lupus Erythematosus: Results of a Twenty-Four-Week, Multicenter, Randomized, Double-Blind, Placebo-Controlled, Parallel-Arm, Phase IIb Study. Arthritis Rheumatol. 2018; 70(2):266-276.
62. Sergott R C, Bennett J L, Rieckmann P, et al. ATON: results from a Phase II randomized trial of the B-cell-targeting agent atacicept in patients with optic neuritis. J Neurol Sci. 2015; 351(1-2):174-178.
63. Gillen J, Richardson D, Moore K. Angiopoietin-1 and Angiopoietin-2 Inhibitors: Clinical Development. Curr Oncol Rep. 2019; 21(3):22.
64. Cho N H, Shaw J E, Karuranga S, et al. IDF Diabetes Atlas: Global estimates of diabetes prevalence for 2017 and projections for 2045. Diabetes Res Clin Pract. 2018; 138:271-281.
65. Giuliari G P. Diabetic retinopathy: current and new treatment options. Curr Diabetes Rev. 2012; 8(1):32-41.
66. Arevalo J F, Garcia-Amaris R A. Intravitreal bevacizumab for diabetic retinopathy. Curr Diabetes Rev. 2009; 5(1):39-46.
67. Kudo Y, lizuka S, Yoshida M, et al. Matrix metalloproteinase-13 (MMP-13) directly and indirectly promotes tumor angiogenesis. J Biol Chem. 2012; 287(46):38716-38728.
68. Kim B, Abdel-Rahman M H, Wang T, Pouly S, Mahmoud A M, Cebulla C M. Retinal MMP-12, MMP-13, TIMP-1, and TIMP-2 expression in murine experimental retinal detachment. Invest Ophthalmol Vis Sci. 2014; 55(4):2031-2040.
69. Lecomte J, Louis K, Detry B, et al. Bone marrow-derived mesenchymal cells and MMP13 contribute to experimental choroidal neovascularization. Cell Mol Life Sci. 2011; 68(4):677-686.
70. van Deemter M, Pas H H, Kuijer R, van der Worp R J, Hooymans J M, Los L I. Enzymatic breakdown of type II collagen in the human vitreous. Invest Ophthalmol Vis Sci. 2009; 50(10):4552-4560.
71. Abu E I-Asrar A M, Mohammad G, Nawaz M I, et al. Relationship between vitreous levels of matrix metalloproteinases and vascular endothelial growth factor in proliferative diabetic retinopathy. PLoS One. 2013; 8(12):e85857.
72. Shahulhameed S, Vishwakarma S, Chhablani J, et al. A Systematic Investigation on Complement Pathway Activation in Diabetic Retinopathy. Front Immunol. 2020; 11:154.
73. Liao D S, Grossi F V, El Mehdi D, et al. Complement C3 Inhibitor Pegcetacoplan for Geographic Atrophy Secondary to Age-Related Macular Degeneration: A Randomized Phase 2 Trial. Ophthalmology. 2020; 127(2):186-195.
74. Wert K J, Mahajan V B, Zhang L, et al. Neuroretinal hypoxic signaling in a new preclinical murine model for proliferative diabetic retinopathy. Signal Transduct Target Ther. 2016; 1.
75. Zou C, Han C, Zhao M, et al. Change of ranibizumab-induced human vitreous protein profile in patients with proliferative diabetic retinopathy based on proteomics analysis. Clin Proteomics. 2018; 15:12.
76. Schori C, Trachsel C, Grossmann J, Zygoula I, Barthelmes D, Grimm C. The Proteomic Landscape in the Vitreous of Patients With Age-Related and Diabetic Retinal Disease. Invest Ophthalmol Vis Sci. 2018; 59(4):Amd31-amd40.
77. Li J, Lu Q, Lu P. Quantitative proteomics analysis of vitreous body from type 2 diabetic patients with proliferative diabetic retinopathy. BMC Ophthalmol. 2018; 18(1):151.
78. Loukovaara S, Nurkkala H, Tamene F, et al. Quantitative Proteomics Analysis of Vitreous Humor from Diabetic Retinopathy Patients. J Proteome Res. 2015; 14(12):5131-5143.
79. Herndndez C, García-Ramírez M, Colomé N, et al. Identification of new pathogenic candidates for diabetic macular edema using fluorescence-based difference gel electrophoresis analysis. Diabetes Metab Res Rev. 2013; 29(6):499-506.
80. Wang H, Feng L, Hu J W, Xie C L, Wang F. Characterisation of the vitreous proteome in proliferative diabetic retinopathy. Proteome Sci. 2012; 10(1):15.
81. Gao B B, Chen X, Timothy N, Aiello L P, Feener E P. Characterization of the vitreous proteome in diabetes without diabetic retinopathy and diabetes with proliferative diabetic retinopathy. J Proteome Res. 2008; 7(6):2516-2525.
82. Kim T, Kim S J, Kim K, et al. Profiling of vitreous proteomes from proliferative diabetic retinopathy and nondiabetic patients. Proteomics. 2007; 7(22):4203-4215.

TABLE 1
Prior Proteomic Studies Including Patients with Proliferative Diabetic Retinopathy (PDR)

| | | | | | | | | No. Proteins | |
|---|---|---|---|---|---|---|---|---|---|
| | Disease | Total | PDR | Control | | Age | | Differentially | Protein |
| Study | Investigated | N | N | Group | Ethnicity | Range | Methods | Expressed | Focus |
| 2018 Zou et | Untreated | 26 | 17 | IMH | China | 53.9 +/− | LC- | 72 | APOC1, |
| al.⁷⁵ | PDR/IV | | | | | 12.5 | MS/MS, | | SERPINA5, |
| | ranibizumab | | | | | | ELISA | | TIMP2, |
| | PDR | | | | | | (CUSABIO) | | KRT1 |
| 2018 Schori | dry | 34 | 10 | ERM | Switzerland | NR | LC- | 142 | CFAI, |
| et al.⁷⁶ | AMD/nAMD/ | | | | | | MS/MS, | | CH3L1, |
| | PDR/ERM | | | | | | sandwich- | | HEM2, |
| | | | | | | | ELISA | | RNAS1, |
| | | | | | | | | | SODC, |
| | | | | | | | | | VEGFR1 |
| 2018 Li et | DMTII PDR | 18 | 9 | IMH | China | NR | LC- | 62 | KNG1, |
| al.⁷⁷ | | | | | | | MS/MS, | | APOF, |
| | | | | | | | elite hybrid | | CA1, |
| | | | | | | | MS | | CAT, |
| | | | | | | | coupled to | | COL1A1, |
| | | | | | | | EASY-nLC | | C2, |
| | | | | | | | | | C4BPA, |
| | | | | | | | | | CFD, |
| | | | | | | | | | CFHR3, |
| | | | | | | | | | CFH, |
| | | | | | | | | | FGA, |
| | | | | | | | | | PON1, |
| | | | | | | | | | VTN |
| 2015 | NPDR/PDR/ | 138 | 79 | none | Finland | NR | LC- | 230 | LUM, |
| Loukovaara | anti-VEGF | | | | | | MS/MS, | | KERA, |
| et al.⁷⁸ | treated PDR | | | | | | elite hybrid | | TLR, |
| | | | | | | | MS | | A2MG, |
| | | | | | | | coupled to | | VTDB, |
| | | | | | | | EASY-nLC | | PRDX2, |
| | | | | | | | | | PRDX6 |
| | | | | | | | | | CATA, |
| | | | | | | | | | ROMO1, |
| | | | | | | | | | C1QB, |
| | | | | | | | | | CFAB, |
| | | | | | | | | | CFAD, |
| | | | | | | | | | CFAH, |
| | | | | | | | | | CFAI, |
| | | | | | | | | | CO2, |
| | | | | | | | | | CO3, |
| | | | | | | | | | CO4B, |
| | | | | | | | | | CO5, |
| | | | | | | | | | CO7, |
| | | | | | | | | | CO8A, |
| | | | | | | | | | CO8B, |
| | | | | | | | | | CO8G, |
| | | | | | | | | | CO9, |
| | | | | | | | | | FHR1, |
| | | | | | | | | | AACT, |
| | | | | | | | | | A1AT, |
| | | | | | | | | | A2AP, |
| | | | | | | | | | ANGT, |
| | | | | | | | | | ANT3, |
| | | | | | | | | | CBG, |
| | | | | | | | | | HEP2, |
| | | | | | | | | | IC1, |
| | | | | | | | | | KAIN, |
| | | | | | | | | | APOA1, |
| | | | | | | | | | APOA2, |
| | | | | | | | | | APOA4, |
| | | | | | | | | | APOB, |
| | | | | | | | | | APOC1, |
| | | | | | | | | | APOC2, |
| | | | | | | | | | APOC3, |
| | | | | | | | | | APOH, |
| | | | | | | | | | APOM, |
| | | | | | | | | | ABLM1, |
| | | | | | | | | | AKAP4, |
| | | | | | | | | | CFA52, |
| | | | | | | | | | CROCC, |
| | | | | | | | | | DYHC2, |
| | | | | | | | | | FAT4, |
| | | | | | | | | | GELS, |
| | | | | | | | | | TT21B, |
| | | | | | | | | | IGHA1, |
| | | | | | | | | | IGHA2, |
| | | | | | | | | | IGHG2, |
| | | | | | | | | | IGHG3, |
| | | | | | | | | | IGHM, |
| | | | | | | | | | MUCB, |
| | | | | | | | | | CO4A2, |
| | | | | | | | | | CO6A3, |
| | | | | | | | | | CORA1, |
| | | | | | | | | | FIBA, |
| | | | | | | | | | FIBB, |
| | | | | | | | | | and |
| | | | | | | | | | FIBG, |
| | | | | | | | | | AMBP, |
| | | | | | | | | | ITIH1, |
| | | | | | | | | | ITIH2, |
| | | | | | | | | | ITIH3, |
| | | | | | | | | | and |
| | | | | | | | | | ITHI4, |
| | | | | | | | | | A1AG1, |
| | | | | | | | | | A1AG2, |
| | | | | | | | | | A1BG, |
| | | | | | | | | | FETUA |
| 2013 | DME wo | 16 | 4 | IMH | Spain | 60 +/− | DIGE, MS, | 25 | APOH, |
| Hernandez | PDR/DME & | | | | | 10 | ELISA | | GSN, |
| et al.⁷⁹ | PDR | | | | | | | | IRBP, |
| | | | | | | | | | TIMP2, |
| | | | | | | | | | PTGDS, |
| | | | | | | | | | DBP |
| 2012 Wang | PDR | 20 | 10 | Cornea | China | 48.8 +/− | 2D-DIGE/ | 29 | DDAH1, |
| et al.⁸⁰ | | | | transplant | | 11.3 | MALDI- | | TUBA1B, |
| | | | | | | | TOF MS/ | | ENO2, |
| | | | | | | | MALDI- | | ACOT7, |
| | | | | | | | TOF | | MDH, |
| | | | | | | | tandem | | PEBP1, |
| | | | | | | | MS | | PEDF, |
| | | | | | | | | | CLU |
| 2008 Gao et | PDR/no DR | 17 | 7 | IMH/ERM/ | US (CA, MA) & | 45.3 +/− | SDS- | 56 | C1QB, |
| al.⁸¹ | | | | RD | Mexico | 10.7 | PAGE, | | C1QC, |
| | | | | | | | nanoLC- | | C1R, |
| | | | | | | | MS/MS | | C2, C3, |
| | | | | | | | | | C4A, |
| | | | | | | | | | C5, C7, |
| | | | | | | | | | C8G, |
| | | | | | | | | | C9, |
| | | | | | | | | | CFB, |
| | | | | | | | | | CFD, |
| | | | | | | | | | CFH, |
| | | | | | | | | | CFI, |
| | | | | | | | | | SERPING1, |
| | | | | | | | | | A2M, |
| | | | | | | | | | F2, F5, |
| | | | | | | | | | FGA, |
| | | | | | | | | | FGB, |
| | | | | | | | | | SERPINA1, |
| | | | | | | | | | SERPINA5, |
| | | | | | | | | | SERPINC1, |
| | | | | | | | | | F12, |
| | | | | | | | | | PLG, |
| | | | | | | | | | KNG1, |
| | | | | | | | | | SERPINF2 |
| | | | | | | | | | AGT, |
| | | | | | | | | | CLSTN1, |
| | | | | | | | | | RBP3, |
| | | | | | | | | | SERPINI1, |
| | | | | | | | | | A1AT, |
| | | | | | | | | | AT3, |
| | | | | | | | | | PRDX1, |
| | | | | | | | | | SOD |
| 2007 Kim et | PDR for 2- | 33 | 19 | MH | Korean | 62.5(32- | (IS)/2- | 415 | VEGF, |
| al.⁸² | DE (8) | | | | | 72) | DE/MALDI- | | IGFBP- |
| | PDR for LC- | | | | | 56.0(52- | MS, nano- | | 2, |
| | MS/MS (11) | | | | | 73) | LC- | | IGFBP3, |
| | | | | | | | MALDI- | | IGFBP- |
| | | | | | | | MS/MS, | | 5, IGF1, |
| | | | | | | | nano-LC- | | IGF2, |
| | | | | | | | ESI- | | HGF, |
| | | | | | | | MS/MS | | CA, |
| | | | | | | | | | OPN, |
| | | | | | | | | | AGT, |
| | | | | | | | | | CLU, |
| | | | | | | | | | ANG, |
| | | | | | | | | | CP, TF, |
| | | | | | | | | | TTR, |
| | | | | | | | | | SERPIN, |
| | | | | | | | | | WIF-1 |

2-DE = two-dimensional gel electrophoresis;
AMD = age-related macular degeneration;
DIGE = differential gel electrophoresis;
DME = diabetic macular edema;
DMTII = diabetes mellitus, type II;
ELISA = enzyme-linked immunosorbent assay;
ERM = epiretinal membrane;
IMH = idiopathic macular hole;
IV = intravenous;
LC = liquid chromatography;
MALDI = matrix-assisted laser desorption/ionization;
MS = mass spectrometry;
nAMD = neovascular age-related macular degeneration;
NPD = non-proliferative diabetic retinopathy;
NR = not recorded;
PDR = proliferative diabetic retinopathy;
RD = retinal detachment;
SDS-PAGE = sodium dodecyl sulphate-polyacrylamide gel electrophoresis;
TOF = time-of-flight mass spectrometer;
VEGF = vascular endothelial growth factor

TABLE 2
Patient Demographics
Diabetic Retinopathy Samples

| | | | | | Surgical | |
|---|---|---|---|---|---|---|
| Patient | Sex | Age* | Eye | Diabetes Mellitus Type | Indication | Phakia |
| 1 | M | 46 | OS | Type I, IDDM | PDR - VH | Pseudophakic |
| 2 | M | 52 | OD | Type II, NIDDM | PDR - TRD/RRD | Phakic |
| 3 | F | 40 | OS | Type II, NIDDM | PDR - TRD | Phakic |
| 4 | M | 31 | OD | Type I, IDDM | PDR - VH | Phakic |
| 5 | M | 49 | OD | Type I, IDDM | PDR - VH | Phakic |
| 6 | F | 39 | OD | Type II, NIDDM | PDR - VH | Phakic |

Control Samples

| | | | | | Surgical | |
|---|---|---|---|---|---|---|
| Patient | Sex | Age* | Eye | Other Diagnoses | Indication | Phakia |
| 7 | M | 67 | OD | Mild BRVO | ERM | Pseudophakic |
| 8 | M | 77 | OD | NIDDM, Type II without | ERM | Pseudophakic |
| | | | | retinopathy | | |
| 9 | F | 85 | OD | Peripapillary CNVM, Mild | ERM | Pseudophakic |
| | | | | BRVO, Mild NPDR, | | |
| | | | | Retinoschisis | | |
| 10 | M | 65 | OD | Choroidal nevus | ERM | Phakic |
| 11 | M | 69 | OD | CME | ERM | Phakic |
| 12 | M | 56 | OD | RRD status post scleral | ERM | Pseudophakic |
| | | | | buckle repair, PVD | | |

IDDM = insulin dependent diabetes mellitus;
PDR = proliferative diabetic retinopathy;
VH = vitreous hemorrhage;
NIDDM = non-insulin dependent diabetes mellitus;
TRD = tractional retinal detachment;
RRD = rhegmatogenous retinal detachment;
BRVO = branch retinal vein occlusion;
ERM = epiretinal membrane;
CNVM = choroidal neovascular membrane;
CME = cystoid macular edema;
PVD = posterior vitreous detachment
*At the time of surgery

1. A method of diagnosing and treating proliferative diabetic retinopathy (PDR) in a patient, the method comprising:
a) obtaining a vitreous sample from an eye of the patient;
b) measuring levels of expression of one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the vitreous sample, wherein decreased levels of expression of the one or more biomarkers selected from interleukin 12 (IL-12p70), thyroid-stimulating hormone (TSH), interleukin-I family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) compared to reference value ranges for a vitreous sample from a control subject, and increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) compared to reference value ranges for a vitreous sample from a control subject indicate that the patient has PDR; and
c) treating the patient for the PDR, if the patient has a positive diagnosis for PDR.

2. The method of claim 1, wherein the levels of expression of at least two, at least three, or at least four biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) are measured in the vitreous sample.

3. The method of claim 1, wherein the levels of expression of interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) are measured in the vitreous sample.

4. The method of claim 1, wherein said treating the patient for the PDR comprises administering a corticosteroid or a vascular endothelial growth factor (VEGF) inhibitor or performing laser surgery or a vitrectomy, or any combination thereof.

5. The method of claim 4, wherein the corticosteroid is triamcinolone or fluocinolone acetonide.

6. The method of claim 4, wherein the VEGF inhibitor is bevacizumab, aflibercept, or ranibizumab.

7. The method of claim 4, wherein the laser surgery is photocoagulation or panretinal photocoagulation.

8. The method of claim 1, wherein said measuring the levels of expression comprises performing mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, fluorescence-activated cell sorting (FACS), or a Western Blot.

9. The method of claim 8, wherein the ELISA is performed using a multiplex ELISA array.

10. A method of monitoring proliferative diabetic retinopathy (PDR) in a patient, the method comprising:
a) obtaining a first vitreous sample from an eye of the patient at a first time point and a second vitreous sample from the eye of the subject later at a second time point;
b) measuring one or more biomarkers in the first vitreous sample and the second vitreous sample, wherein the biomarkers are selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL); and
c) analyzing the levels of expression of the one or more biomarkers in conjunction with respective reference value ranges for said biomarkers, wherein detection of decreased levels of expression of the one or more biomarkers selected from interleukin 12 (IL-12p70), thyroid-stimulating hormone (TSH), interleukin-I family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and detection of increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening, and detection of increased levels of expression of the one or more biomarkers selected from interleukin 12 (IL-12p70), thyroid-stimulating hormone (TSH), interleukin-I family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and decreased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL in the second vitreous sample compared to the first vitreous sample indicate that the patient is improving.

11. A method of monitoring efficacy of a treatment of a patient for proliferative diabetic retinopathy (PDR), the method comprising:
a) obtaining a first vitreous sample from the patient before the patient undergoes the treatment and a second vitreous sample from the subject after the patient undergoes the treatment;
b) measuring one or more biomarkers in the first vitreous sample and the second vitreous sample, wherein the biomarkers are selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), tumor necrosis factor receptor superfamily member 9 (4-1BB), vascular endothelial growth factor (VEGF), galectin-2 (LGALS2), transmembrane activator and CAML interactor (TACI), monocyte chemoattractant protein 1 (MCP-1), angiopoietin 2 (ANG-2), interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL); and
c) evaluating the efficacy of the treatment, wherein detection of decreased levels of expression of the one or more biomarkers selected from interleukin 12 (IL-12p70), thyroid-stimulating hormone (TSH), interleukin-I family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and detection of increased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL) in the second vitreous sample compared to the first vitreous sample indicate that the patient is worsening or not responding to the treatment, and detection of increased levels of expression of the one or more biomarkers selected from interleukin 12 (IL-12p70), thyroid-stimulating hormone (TSH), interleukin-1 family member 6 (IL-1 F6), fibroblast growth factor 6 (FGF-6), bone morphogenetic protein 7 (BMP-7), interleukin 1 beta (IL-1b), follicle-stimulating hormone (FSH), tafazzin (TAZ), interleukin 34 (IL-34), carcinoma antigen 15-3 (CA15-3), albumin (ALB), cadherin-6 (CDH6), semaphorin 6D (SEMA6D), tenascin R (TNR), and tumor necrosis factor receptor superfamily member 9 (4-1BB) and decreased levels of expression of the one or more biomarkers selected from vascular endothelial growth factor (VEGF), galectin-2 (LGALS2), transmembrane activator and CAML interactor (TACI), monocyte chemoattractant protein 1 (MCP-1), angiopoietin 2 (ANG-2), Interferon gamma-induced protein 10 (IP-10), galectin-3 (LGALS3), plasminogen activator inhibitor-1 (PAI-1), C—C motif chemokine ligand 4 (MIP-1b), cluster of differentiation 97 (CD97), platelet-derived growth factor having 2 A subunits (PDGF-AA), regenerating family member 4 (REG4), nucleosome assembly protein 1 like 4 (NAP-2), ferritin, matrix metallopeptidase 13 (MMP-13), cluster of differentiation 99 (CD99), B cell maturation antigen (BCMA), cathepsin S (CTSS), T cell immunoglobulin mucin 3 (TIM-3), midkine (MDK), glycoxalase II, and mannose-binding lectin (MBL in the second vitreous sample compared to the first vitreous sample indicate that the patient is improving.

12. The method of claim 11, further comprising altering the treatment if the patient is worsening or not responding to the treatment.

13-19. (canceled)

20. A method of treating a subject for proliferative diabetic retinopathy, the method comprising administering a therapeutically effective amount of pimagedine, atacicept, or an ANG-2 inhibitor to the subject.

21. The method of claim 20, wherein the ANG-2 inhibitor is trebananib, tebastanib, or MEDI3617.

22. The method of claim 20, wherein the pimagedine, atacicept, or ANG-2 inhibitor is administered locally to the eye.

23. The method of claim 22, wherein the pimagedine, atacicept, or ANG-2 inhibitor is administered locally to the retina.

24. The method of claim 20, wherein the pimagedine, atacicept, or ANG-2 inhibitor is administered intravitreally, intravenously, subcutaneously, or orally.

25. The method of claim 20, wherein multiple therapeutically effective doses of the pimagedine, atacicept, or ANG-2 inhibitor are administered to the subject.

26. The method of claim 25, wherein the pimagedine, atacicept, or ANG-2 inhibitor are administered daily or intermittently.

27-30. (canceled)
